(19) United States
(12) Patent Application Publication
(10) Pub. No.: US 2026/0345760 A1
(43) Pub. Date: Nov. 5, 2026

(54) COMPOSITIONS AND METHODS FOR RAISING IMMUNE RESPONSES TO GRAM-NEGATIVE BACTERIA

(71) Applicant: Administrators of the Tulane Educational Fund, New Orleans, LA (US)

(72) Inventors: Jay K. KOLLS, New Orleans, LA (US); Akhilesh Kumar SRIVASTAVA, New Orleans, LA (US); Joseph P. HOFFMANN, New Orleans, LA (US); Janet E. MCCOMBS, New Orleans, LA (US); Elizabeth B. NORTON, New Orleans, LA (US)

(73) Assignee: Administrators of the Tulane Educational Fund, New Orleans, LA (US)

(21) Appl. No.: 19/108,116

(22) PCT Filed: Aug. 30, 2023
(86) PCT No.: PCT/US2023/073208

§ 371 Date: Feb. 28, 2025

Related U.S. Application Data
(60) Provisional application No. 63/402,303, filed on Aug. 30, 2022.

(51) Int. Cl. A61K 39/108; A61K 39/00; A61K 39/39; A61P 37/04

(52) U.S. Cl. CPC A61K 39/0266; A61K 39/39; A61P 37/04; A61K 2039/541; A61K 2039/543; A61K 2039/544; A61K 2039/55527

(57) ABSTRACT

It has been discovered that immunogens of conserved proteins from Gram-negative bacteria (GNB) co-administered with IL1α provoke a strong T-cell Lung Burdens response to the bacteria from which the immunogens were derived. The compositions further provoke cross reactivity with other GNB having homologs of the conserved proteins. This T-cell response is particularly useful in raising effective immune responses to bacterial that infect mucosal surfaces. The disclosure provides compositions and methods for raising robust immune responses to Gram-negative bacteria.

### CROSS-REFERENCE TO RELATED APPLICATIONS

This application priority to, and the benefit of, U.S. Provisional Patent Application No. 63/402,303, filed Aug. 30, 2022, the contents of which are incorporated herein by reference for all purposes.

### STATEMENT OF FEDERAL FUNDING

This invention was made with government support under Grant Nos. R35HL139930 and R01AI149119 awarded by the National Institutes of Health. The Government has certain rights in this invention

### BACKGROUND OF THE INVENTION

Growing antimicrobial resistance to common antibiotics is a significant health issue worldwide. Tens of thousands of deaths are estimated to occur each year due to resistance to available antibiotics. Carbapenems, for example, are widely relied on for their broad-spectrum antibacterial activity against both Gram-positive and Gram-negative bacteria, see, e.g., Codjoe and Donkor, Med Sci (Basel). 2018 March; 6(1):1. doi: 10.3390/medsci6010001, but the use of this class of antibiotics is being threatened by the rapid rise of carbapenem-resistant Enterobacteriaceae, or “CRE,” such as Klebsiella pneumoniae. Id. A study of eight medical centers in the New York area in 2017, for example, reported that almost ten percent of patients infected with K. pneumoniae were CRE (Satlin et al., Antimicrob Agents Chemother. 2017 Mar. 24; 61(4):e02349-16. doi: 10.1128/AAC.02349-16).

Vaccines are a cost-effective medical intervention, and one of the great advances in promoting human health. Unfortunately, many vaccines suffer from weak or limited immunogenicity. This is particularly true for killed organism vaccines and for sub-unit vaccines, which are generally poor inducers of adaptive immunity and generate a primarily humoral response, with little induction of cell-mediated activity. See, e.g., Lee and Nguyen, Immune Network, 2015, 15(2):51-57, doi.org/10.4110/in.2015.15.2.51; Schijns and Lavelle, Expert Review of Vaccines, 2014, 10:4, 539-550, DOI: 10.1586/erv.11.21. The immunogenicity of vaccines can be enhanced by use of adjuvants, substances which enhance a subject's immune response to a vaccine. Unfortunately, many substances that strongly increase immune response in animals, such as Freund's Complete Adjuvant, are also toxic and only a few adjuvants have been approved for use in humans. The most commonly used adjuvant is “alum,” which denotes any of several aluminum salts that are used as an adjuvant in over 80% of currently approved vaccines. Unfortunately, alum adjuvants tend to enhance humoral (antibody) responses but are poor inducers of cell-mediated responses.

Gram-negative bacteria secrete outer membrane vesicles (“OMVs”), which are sections of outer membrane that separate from the cell and which individually encapsulate a portion of periplasmic space. See, e.g., Klimentova and Stulik, Microbiological Res, 2015, 170:1-9, doi.org/10.1016/j.micres.2014.09.006. OMVs contain constituents of the outer membrane, such as lipopolysaccharide, phospholipids and proteins, and may contain virulence factors and other cytosolic proteins. Id. OMVs of meningococcus have been explored as adjuvants for meningococcal vaccines since at least 2011 (see, e.g., Sanders and Feavers, Expert Rev Vaccines. 2011 March; 10(3):323-34. doi: 10.1586/erv.11.10). A report in 2014 showed meningococcus engineered to produce OMVs with a less toxic form of lipopolysaccharide enhanced immune response to both meningococcal antigens and to tetanus toxoid. Nagaputra et al., Clin. Vaccine Immunol., 2014, 21(2):234-242. Gram-negative bacteria also contain outer membrane proteins, including the membrane spanning proteins known as porins. Porins act as pores through which molecules can diffuse. Reviewed in, e.g., Vergalli, et al., Nat Rev Microbiol, 2020, 18:164-176; doi.org/10.1038/s41579-019-0294-2; Yen, et al., Biochimica et Biophysica Acta-Biomembranes, 2002, 1562(1-2):6-31; doi.org/10.1016/S0005-2736(02)00359-0.

A need remains in the art for adjuvants which can elicit enhanced cell-mediated immunity and humoral responses to immunogens derived from Gram-negative bacteria, such as Enterobacteriaceae, and against CRE such as Klebsiella pneumoniae in particular. Surprisingly, the invention fills these and other needs.

### BRIEF SUMMARY OF THE INVENTION

In some embodiments, the invention provides immunogenic compositions comprising (a) at least one immunogen from a Gram-negative bacterium (“GNB”) and (b) IL1α. In some embodiments, the GNB is an Enterobacteriae. In some embodiments, the GNB is Klebsiella pneumoniae. In some embodiments, the at least one immunogen from the GNB and the IL1α are admixed. In some embodiments, the composition further comprises an Omp from a second GNB. In some embodiments, the at least one immunogen from the GNB and the IL1α are expressed in a fusion protein. In some embodiments, the at least one immunogen from said GNB is an outer membrane vesicle. In some embodiments, the at least one immunogen from said GNB is an outer membrane protein (“Omp”). In some embodiments, the Omp is a porin. In some embodiments, the Omp is from K. pneumoniae. In some embodiments, the Omp is one or more of OmpX, OmpC, OmpW, and Omplolb. In some embodiments, the composition comprises two or more of OmpX, OmpC, OmpW, and Omplolb. In some embodiments, the immunogenic composition is a fusion protein comprising (a) one or more of OmpX, OmpC, OmpW, and Omplolb, and (b) IL1α. In some embodiments, one Omp of said one or more of OmpX, OmpC, OmpW, and Omplolb is closest on the fusion protein to the IL1α and the one Omp closest on the fusion protein to the IL1α is linked to the IL1α through a peptide linker. In some embodiments, the peptide linker is a GS linker. In some embodiments, the porin is OmpX. In some embodiments, the OmpX is expressed in a fusion protein with IL1α. In some embodiments, the OmpX is linked to said IL1α by a GS linker. In some embodiments, the fusion protein has the sequence of SEQ ID NO:1.

In another group of embodiments, the invention provides methods of increasing a subject's immune response to an immunogen from a Gram-negative bacterium (“GNB”), said method comprising co-administering to said subject (a) an effective amount of said immunogen and (b) an effective amount of IL1α. In some embodiments, the effective amount of immunogen and said effective amount of said IL1α are mixed to form a single composition prior to said co-administration. In some embodiments, the composition further comprises a stabilizer, a buffer, or both a stabilizer and a buffer. In some embodiments, the composition is lyophilized. In some embodiments, the lyophilized composition is reconstituted prior to said co-administration. In some embodiments, the GNB immunogen is from an Enterobacteriae bacterium. In some embodiments, the GNB immunogen is from Klebsiella pneumoniae. In some embodiments, the GNB immunogen is an outer membrane vesicle. In some embodiments, the GNB immunogen is an outer membrane protein (“Omp”). In some embodiments, the Omp is a K. pneumoniae Omp. In some embodiments, the Omp is OmpX, OmpC, OmpW, and Omplolb. In some embodiments, the composition comprises a second GNB immunogen, wherein the second GNB immunogen is a second Omp selected from OmpX, OmpC, OmpW, and Omplolb. In some embodiments, the co-administration is by administering a fusion protein of (a) one or more of OmpX, OmpC, OmpW, and Omplolb and (b) IL1α. In some embodiments, the one of said one or more of OmpX, OmpC, OmpW, and Omplolb is closest to said IL1α on said fusion protein and said closest of said OmpX, OmpC, OmpW, and Omplolb is linked to said ILlu through a peptide linker. In some embodiments, the peptide linker is a GS linker. In some embodiments, the Omp is OmpX. In some embodiments, the coadministration of OmpX and ILlu is by administering a fusion protein of OmpX and IL1α. In some embodiments, the fusion protein comprises OmpX linked to said IL1α through a peptide linker. In some embodiments, the peptide linker is a GS linker. In some embodiments, the fusion protein has the sequence of SEQ ID NO:1. In some embodiments, the co-administration is intranasal, intrapulmonary, intraperitoneal, subcutaneous, intramuscular, or intracavity. In some embodiments, the intracavity co-administration is intravaginal. In some embodiments, the co-administration is intranasal or intrapulmonary.

### BRIEF DESCRIPTION OF THE DRAWINGS

FIG. 1A is a cartoon showing immunization and challenge schedule with KP-396 (K1 strain) of Klebsiella pneumoniae. FIG. 1B. FIG. 1B is a graph showing bacterial burden post 24 hours of infection with K1 strain of K. pneumoniae, CFU in Lung, determined in wild type C57BL/6 mice immunized intrapulmonarily with vehicle (n=2), OmpX+LTA1 (n=4), and in Il1r1KO C57BL/6 mice (n=4) immunized intrapulmonarily with OmpX+LTA1. FIG. 1C. FIG. 1C is a graph showing a flow cytometry analysis for IL17A cell population in lung single cells gated on CD3+CD4+TCRb+. The analysis shows a high number of IL17A+ cells in WT C57BL/6 mice compared to Il1r1KO mice.

FIG. 2A is a graph showing the bacterial burden, in CFU, in the lungs of mice post 24 hours of infection with K1 strain of Klebsiella pneumoniae, in wild type C57BL/6 mice immunized intrapulmonarily with Vehicle (n=6), OmpX+LTA1 (n=8) or in Casp1-KO C57BL/6 mice (n=10) immunized intrapulmonarily with OmpX+LTA1. These data show no significant difference in vaccine efficacy as measured by lung CFU between OmpX+LTA1 immunized Casp1-KO and wild type C57BL/6 mice. FIG. 2B. FIG. 2B is a graph showing a flow cytometry analysis for IL17A cell population in lung single cells gated on CD3+CD4+TCRb+, showing a high number of IL17A+ cells in WT C57BL/6 and Casp1-KO mice immunized intrapulmonarily with OmpX+LTA1 compared to the vehicle control WT C57BL/6 mice.

FIG. 3A is a graph showing the bacterial burden in CFUs in the lungs of mice immunized with OmpX+rIL1α (n=3), with OmpX+rIL1β (n=3), or with PBS as a vehicle control, 24 hours after infection with K. pneumoniae K1 strain. FIG. 3B. FIG. 3B is a graph showing the bacterial burden in CFUs in the spleens of mice immunized with OmpX+rIL1α (n=3), with OmpX+rIL1β (n=3), or with PBS as a vehicle control, 24 hours after infection with K. pneumoniae K1 strain. The OmpX+rIL1α (n=3) and OmpX+rIL1β (n=3) data showed significant vaccine efficacy as measured by lung CFU compared to PBS controls. (* p<0.05, ANOVA with multiple comparisons.)

FIG. 4A is a visual depiction of a plan for creating a fusion peptide of OmpX linked to IL-1α. FIG. 4B. FIG. 4B is a photograph of a SDS-PAGE analysis showing the purity and molecular weight of a fusion protein FIG. 4C. FIG. 4C sets forth the sequence (SEQ ID NO:1) of an exemplar OmpX-IL-1α fusion protein in which OmpX is linked to IL-1α through an exemplar “GS” peptide linker (SEQ ID NO:2, underlined).

FIG. 5A is a graph of the bacterial burden, in CFUs, in the lungs of C57BL/6 mice immunized with an exemplar OmpX-IL-1α fusion protein (“IL-1α-OmpX FP”) or with PBS, measured 24 hours after infection with K. pneumoniae K1 strain. FIG. 5B. FIG. 5B is a graph of the bacterial burden, in CFUs, in the spleens of C57BL/6 mice immunized with an exemplar OmpX-IL-1α fusion protein (“IL-1α-OmpX FP”) or with PBS, measured 24 hours after infection with K. pneumoniae K1 strain. The mice immunized with the fusion protein had a significant reduction in lung and spleen bacterial burden compared to mice immunized with PBS. (* p<<0.0001, unpaired t test).

FIG. 6 is a Uniform Manifold Approximation and Projection (“UMAP”) plot of lung Th17 cells elicited by IL1α administered with OmpX, either as an admixture or as a fusion protein. Similar types of lung Th17 cells (dark gray) are elicited by IL1α as with LTA1 (middle row) that are unique from naive spleen cells (lighter gray).

FIG. 7A is a representative UMAP plot of Il1r1 expression in CD4+ cells from naïve spleens (top) and OmpX+LTA1 immunized lungs (bottom). FIG. 7B is a graph showing the Log 2 expression levels of Il1r1 from lung CD4+ T cells (lung) and naïve splenic CD4+ T cells (spleen) shown in FIG. 7A. FIG. 7C is a graph of concentrations of GM-CSF in the serum, BAL fluid, and lung homogenate of immunized and unimmunized mice. Data are presented as the mean+/−SEM, n=3 mice per group. FIG. 7D is a graph of concentrations of IL-17A in the same fluids of the same mice as described for FIG. 7C. FIG. 7E is a graph of concentrations of IL-6 in the same fluids of the same mice as described for FIG. 7C. FIG. 7F is a graph of concentrations of IL-27 in the same fluids of the same mice as described for FIG. 7C. FIG. 7G is a graph of concentrations of IL-1β in the same fluids of the same mice as described for FIG. 7C. FIG. 7H is a graph of concentrations of MCP-1 in the same fluids of the same mice as described for FIG. 7C. FIG. 7I is a graph of concentrations of IL-t2p70 in the same fluids of the same mice as described for FIG. 7C. FIG. 7J is a graph of concentrations of TFN-β in the same fluids of the same mice as described for FIG. 7C. FIG. 7K is a graph of concentrations of IFNγ in the same fluids of the same mice as described for FIG. 7C. FIG. 7L is a graph of concentrations of TNF in the same fluids of the same mice as described for FIG. 7C. FIG. 7M is a graph of concentrations of IL-1α in the same fluids of the same mice as described for FIG. 7C. FIG. 7N is a graph of concentrations of IL-1β in the same fluids of the same mice as described for FIG. 7C. Data for FIGS. 7C-N were analyzed using the unpaired student's T test. *, p<0.05; **, p<0.01; ***, p<0.001.

FIG. 8A is a graph showing the quantification of CD4+ T cells in the lungs of vaccinated and unvaccinated wild-type and II1r1^{-/-} mice as measured in flow cytometry. FIG. 8B is a graph showing the quantification of B cells in the lungs of the same mice as described in FIG. 8A. FIG. 8C is a graph showing the quantification of Th17 cells in the lungs of the same mice as described in FIG. 8A. FIG. 8D is a graph showing median fluorescent intensity of FITC stained IL-17A in vaccinated and control mice. FIG. 8E is a graph showing IgA titers specific to heat killed K. pneumoniae obtained from the supernatant of homogenized lungs in each group described in FIG. 8A. FIG. 8F is a graph showing area under the curve analysis of data displayed in FIG. 8E. FIG. 8G is a graph showing log transformed bacterial burdens in the lungs 24 h post-challenge in each group, measured in a cfu plating assay. Data are represented as mean+/−SEM, n=7-9 mice per group. Statistical differences were determined using a one-way ANOVA. *, p<0.05; **, p<0.01; ***, p<0.001; ****, p<0.0001. N=6-8 mice per group. FIG. 8H is a graph showing log transformed bacterial burdens in the spleen of the same mice as described in the description of FIG. 8G.

FIG. 9A is a graph showing the quantification of CD4+ T cells in lungs from immunized and from unimmunized Casp1^{-/-} and wildtype C57Bl/6 mice. FIG. 9B is a graph showing the quantification of B cells in the lungs of the same mice as described for FIG. 9A. FIG. 9C is a graph showing the quantification of Th17 cells in the lungs of the same mice as described for FIG. 9A. FIG. 9D is a graph showing log transformed bacterial burdens in the lungs of the animals 24 h post-challenge, determined using cfu plating assays. FIG. 9E is a graph showing log transformed bacterial burdens in the spleen 24 h post-challenge, determined using cfu plating assays. FIGS. 9A-9E: Data are presented as mean+/−SEM, n=6-10 mice per group. Statistical differences were determined using a one-way ANOVA. **, p<0.01; ***, p<0.001; ****, p<0.0001.

FIG. 10A is a graph showing the quantification of CD4+ T cells in lungs from antibody- and isotype control-treated mice to investigate whether antibody neutralization of IL1α or IL1β prevents vaccine-mediated protection and Th17 cell generation. FIG. 10B is a graph showing the quantification of B cells in lungs from the same mice as described for FIG. 10A. FIG. 10C is a graph showing the quantification of Th17 cells in lungs from the same mice as described for FIG. 10A. FIG. 10D is a graph showing ELISPOT results measuring IL-17A secreting cells after overnight stimulation with OmpX. Data are displayed as spot counts per 10⁵ plated cells from lung homogenate, n=3-4 mice per group. FIG. 10E is a graph showing the serum IgG titers specific to OmpX from each vaccinated group shown in FIG. 10D. FIG. 10E is a graph showing area under the curve (“AUC”) of the data depicted in FIG. 10E. FIG. 10F is a graph showing log transformed bacterial burdens in the lungs from the animals 24 h post-challenge, determined using cfu plating assays. FIG. 10G is a graph showing log transformed bacterial burdens in the spleens of the animals as described in FIG. 10F. FIGS. 10A-H: Data are presented as mean+/−SEM, n=6-9 mice per group. Statistical differences were determined using a one-way ANOVA. *, p<0.05; **, p<0.01; ***, p<0.001; ****, p<0.0001.

FIG. 11A is a graph showing log transformed bacterial burdens in the lungs of C57Bl/6 mice immunized as indicated on the graph, 24 h post-challenge. FIG. 11B is a graph showing log transformed bacterial burdens in the spleen of C57Bl/6 mice immunized as indicated on the graph, 24 h post-challenge. FIG. 11C is a graph showing serum IgG titers specific to OmpX from each vaccinated group as measured in ELISA. FIG. 11D is a graph showing results from an ELISPOT measuring IL-17A secreting cells after overnight stimulation with OmpX. Data are displayed as spot counts per 10⁵ plated cells from lung homogenate.

FIG. 12A is a graph showing the quantification of CD4+ T cells in lungs from Il1a^{Δ559,1} and wildtype C57Bl/6 mice vaccinated via oropharyngeal aspiration with OmpX+LTA1 or PBS (N=4-5 per group). One week following immunization mice were challenged with K. pneumoniae as described. FIG. 12B is a graph showing the quantification of B cells in lungs from the same mice as described for FIG. 12A. FIG. 12C is a graph showing the quantification of Th17 cells in lungs from the same mice as described for FIG. 12A. FIG. 12D presents two graphs, the first showing anti-OmpX serum IgG titers and the second showing AUC analysis of the data presented in the first. Decreased amounts of IL-1α did not impact antibody titers in vaccinated groups. FIG. 12E presents two graphs, the first showing anti-OmpX serum IgA titers and the second showing AUC analysis of the data presented in the first. Decreased amounts of IL-1α did not impact antibody titers in vaccinated groups. FIG. 12F is a graph showing the log transformed bacterial burdens in the lungs 24 h post challenge in each group. Though there was high variability, immunized IL-1α hypomorph mice still had protection from challenge demonstrated by reduced bacterial burdens. FIG. 12G is a graph showing the log transformed bacterial burdens in the spleen 24 h post challenge in each group. Though there was high variability, immunized IL-1α hypomorph mice still had protection from challenge demonstrated by reduced bacterial burdens, particularly in the spleen. FIG. 12H. Levels of IL-1α were measured in the serum of unvaccinated Il1a^{Δ559,1} and wildtype mice before and after challenge. IL-1α levels in the lung homogenate were taken from each group post infection (N=2 per group). FIG. 12H is a graph presenting the results. FIG. 12I is a graph showing IL-1α levels in lung homogenate taken from each group described in FIG. 12H post infection (N=2 per group). IL-1α hypomorphs had similar levels of IL-1α in the lungs when compared to wildtype mice. This suggests that IL-1α hypomorphs are a poor model for IL-1α depletion.

### DETAILED DESCRIPTION

### Introduction

Surprisingly, we have now found that a member of the interleukin IL-1 family, IL-1α, strongly potentiates the immune response to immunogens from Gram-negative bacilli “GNB.” GNB have proved able to rapidly develop resistance to new antibiotics. As stated in a recent article: “The dearth of antibiotic candidates against Gram-negative bacteria and the rise of antibiotic resistance create a global health concern. The challenge lies in the unique Gram-negative bacterial outer membrane that provides the impermeable barrier for antibiotics and sequesters antigen presentation.” Li, et al., Science Advances, 2023, 9(34); doi: 10.1126/sciadv.adg9601. The ability of GNB to rapidly become resistant to new antibiotics and to sequester antigen presentation makes it important to find new methods for reducing morbidity and mortality caused by GNB, and particularly by the Enterobacteriaceae. Thus, vaccines against GNB, and particularly the Enterobacteriaceae, are urgently needed.

As reported in the Examples below, IL-1α was found to strongly potentiate the immune response to an exemplar immunogen from an exemplar Gram-negative bacterium and exemplar Enterobacteriaceae, K. pneumoniae. Further, the immune response induced was not only a strong antibody response, but also, and more importantly for the exemplar bacterium, a strong response by a population of lung cells known as Th17 cells, tissue resident memory (“TRM”) cells that produce the pro-inflammatory cytokine IL-17. The use of an adjuvant is required to elicit Th17 responses in the lung. The results of animal studies reported herein demonstrated that, unlike the common adjuvant alum, which primarily raises a humoral (antibody) response to antigens with which it is co-administered, the use of IL-1α surprisingly caused the subjects to produce Th17 T cell responses against the Gram-negative bacterium from which the antigen was derived, while not diminishing antibody responses.

This result is surprising, in part, because previous reports on the use of IL-1α as an adjuvant to provoke an immune response against an allergen or as a vaccine adjuvant against influenza antigens, the presence of the IL-1α appears to have elicited high levels of Th1 or Th2 cells, indicating that in those studies, the IL-1α polarized T cells along the Th1 or Th2 pathways, rather than the Th17 pathway needed to provide a robust immune response to bacterial pathogens. The basis for this difference in polarization of the T cell response is not known, which is one reason it could not be predicted from the previous reports. Without wishing to be bound by theory, however, it is believed that some characteristic of a bacterial immunogen in contrast to the viral and allergen antigens previously used resulted in the surprisingly different immune response seen in the studies reported herein.

Thus, the immunogenic compositions of the invention are surprisingly useful for use in embodiments in which the practitioner wishes to produce an immune response to a bacterial pathogen that infects the lungs, such as K. pneumoniae. More generally, the immunogenic compositions of the invention are surprisingly useful for use in embodiments in which the practitioner wishes to employ a mucosal adjuvant. Many mucosal adjuvants have been reported, but many, if not most, elicit self-immunogenicity or raise safety concerns due to their toxic activity. It is believed that the immunogenic compositions of the invention are free of these concerns in addition to their advantageously raising the Th17 cell response necessary to protect against a GNB challenge.

The present invention arose from an unexpected observation. The present inventors were investigating the use of the A1 domain of heat-labile enterotoxin from E. coli (the A1 domain of this enterotoxin is sometimes referred to herein as “LTA1”) as a mucosal adjuvant for bacterial antigens. Heat-labile Escherichia coli enterotoxin (“LT”) is known to be a powerful mucosal adjuvant, and it was tested as an adjuvant in an intranasal flu vaccine. In clinical trials, however, several participants developed Bell's palsy, and use of LT as an adjuvant for vaccines for intranasal administration was deemed inadvisable. See, e.g., Lewis, et al., PLoS One. 2009; 4(9): e6999; doi: 10.1371/journalpone.0006999.

Bacterial antigens admixed with E. coli LTA1 generated pulmonary CD4+Th17 cells and IgG/IgA-producing B cells that provide vaccine protection against bacterial pneumonia such as K. pneumoniae in a serotype independent fashion. This protection requires the generation of lung TRM cells. We found that these cells express high levels of the IL-1 receptor, IL-1R1, which is a receptor for IL-1β and IL-1α. Prior work has shown that LTA1 can activate the inflammasome and processing of IL-1β, and we thought that IL-1β might therefore be required for generation of TRM cells. In support of this, vaccine efficacy was compromised in mice that lack expression of IL-1R1. But, the studies reported in the Examples, below, surprisingly showed that vaccine efficacy was maintained in mice deficient in caspase-1, a molecule that is required for the processing and secretion of IL-1$. These data indicated that the protection raised by the use of LTA1 as an adjuvant was due to the presence of IL-1α. This unexpected result led us to the studies noted above in which we tested IL-1α as an adjuvant for an exemplar immunogen from K. pneumoniae, a bacterium which is an exemplar Enterobacteriaceae and, like all Enterobacteriaceae, also a Gram-negative bacterium.

For convenience of reference, Gram-negative bacteria are sometimes referred to herein by the abbreviation “GNB.” Immunogens derived from a Gram-negative bacterium against which the practitioner wishes to raise an immune response are sometimes referred to herein as “GNB-immunogens” or “GNB-antigens.” As discussed further below, in some embodiments, the GNB-immunogen may be an outer membrane vesicle, or “OMV,” from a Gram-negative bacterium against which the practitioner wishes to raise an immune response (such a GNB is sometimes referred to herein as a “GNB of interest”). In some preferred embodiments, the GNB-immnunogen may be an outer membrane protein, or “Omp,” from a GNB of interest. In some embodiments, the Omps in the inventive compositions and methods may be include two or more Omps from a GNB of interest, to reduce the chance that the GNB of interest will be able to evade or evolve around the immune response elicited by the inventive compositions. In some embodiments, the Omps in the inventive compositions and methods may be from two or more GNBs of interest.

In some embodiments, the Omps used in the inventive compositions and methods are porins. Porins are well-studied beta barrel proteins that cross the membranes of both bacterial cells and of eukaryotic cells. Reviewed in, e.g., Vergalli, et al., Nat Rev Microbiol, 2020, 18:164-176; doi.org/10.1038/s41579-019-0294-2; Yen, et al., Biochimica et Biophysica Acta-Biomembranes, 2002, 1562(1-2):6-31; doi.org/10.1016/S0005-2736(02)00359-0.

The porins of Enterobacteriaceae are highly conserved, and work from the laboratories of some of the present inventors using an exemplar porin, OmpX, as an immunogen has previously demonstrated that, for example, K. pneumoniae OmpX-specific T cells also cross react with bacterial cells of multiple other species of Enterobacteriaceae, including Enterobacter spp. and Acinetobacter baumanii. Given this, it is expected that T cells generated with other porins from other Enterobacteriaceae will cross react with, and provide protection against, other members of the Enterobacteriaceae expressing homologs of the same porins, when those porins are adjuvanted with IL1α. Further, given the results with an exemplar porin, it is also expected that T cells generated with conserved proteins from K. pneumoniae or from other Enterobacteriaceae other than porins will cross react with, and provide protection against, other members of the Enterobacteriaceae expressing homologs of the same protein, when those proteins are adjuvanted with IL1α. GenBank contains an extraordinarily large database of detailed information on members of the Enterobacteriaceae, including what GenBank terms a “genome data package,” setting forth the genome, transcript, protein sequence, annotation, and data report for member of the Enterobacteriaceae listed by species and, where applicable, by strain or serovar. The proteins of the Enterobacteriaceae are therefore known and readily compared to find homologs shared by members of the Enterobacteriaceae against which the practitioner wishes to provide protection.

In some embodiments, the GNB-immunogen may be an immunogen from a member of the Enterobacteriaceae. In some embodiments, the immunogen from a member of the Enterobacteriaceae may be an immunogen from a Klebsiella bacterium. In some embodiments, the immunogen from a K. pneumoniae bacterium may be an OMV. In some embodiments, the immunogen from a K. pneumoniae bacterium may be one or more Omps, such as OmpX. In some embodiments, the Omp or Omps from a K. pneumoniae bacterium may be one or more porins.

Compositions comprising a GNB-immunogen and IL-1α are expected to provide surprisingly superior compositions to raise immune responses against Gram-negative pathogens in which a cellular response is expected to be useful; such compositions are expected to be useful as adjuvants for vaccines against GNB for which alum or other current adjuvants that primarily raise humoral response do not provide an effective adjuvant response, or to further potentiate the adjuvant response provided by alum or other current adjuvants which primarily raise a humoral response. Similarly, compositions comprising an immunogen from a member of the Enterobacteriaceae and IL-1α are expected to provide surprisingly superior compositions to raise immune responses against that member of the Enterobacteriaceae, while compositions comprising an immunogen from K. pneumoniae and IL-1α are expected to provide surprisingly superior compositions to raise immune responses against K. pneumoniae.

In animal studies underlying the present disclosure, mice were administered a fusion protein of an exemplar GNB-OMP, OmpX, from K. pneumoniae, fused through a short peptide linker to IL-1α. As reported in the Examples, animals to which this exemplar fusion protein was administered were then challenged with K. pneumoniae. The animals vaccinated with the exemplar fusion protein showed markedly lower levels of bacterial burden compared to animals vaccinated with phosphate-buffered saline as a control.

Without wishing to be bound by theory, it is believed that enhancement of a subject's immune response is due to antigen presenting cells, such as dendritic cells, encountering the IL-1α and the GNB-OMP antigen together, which either does not happen or is significantly attenuated if the IL-1α and the antigen are administered at different sites. It is noted that IL-1α is naturally produced by some tissues, such as cells of the human epidermis. It is expected that the immunogenic compositions of the invention are not administered into the epidermis and produce an immune response in the subject when administered to other tissues, such as by injection into a muscle or by intranasal administration into the nasal passages.

The GNB immunogen can be co-administered with IL-1α by admixing the two. For example, for intranasal administration, the GNB immunogen can be administered in the same liquid, powder or other form in which the IL-1α is being administered. In preferred embodiments, however, the GNB immunogen is administered as a fusion protein with IL-1α. Conveniently, the GNB immunogen and IL-1α are linked through a peptide linker.

The compositions comprising the GNB antigen and IL-1α may further contain pharmaceutically acceptable excipients suitable for maintaining desired properties for the intended route of administration, such as pH, salt content, anti-caking and other common characteristics familiar to those in the art of formulating vaccine compositions for use in various routes of administration.

In some embodiments, GNB antigen and IL-1α are administered orally, intranasally, or by injection. Immunogenic compositions of the invention are also expected to be effective when administered by other conventional routes. The administration may be, for example, intravenous, intraperitoneal, intramuscular, intracavity. The route of administration for an immunogenic composition may be selected based on where a particular GNB occurs during an infection. K. pneumoniae, for example, infects the lungs, and intranasal administration of immunogenic compositions are expected to raise an immune response in the lungs.

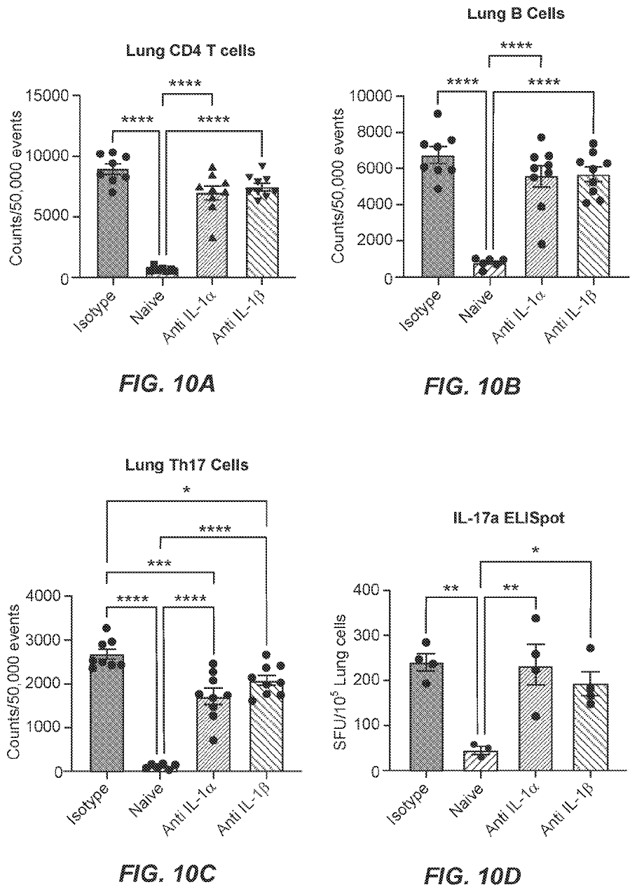

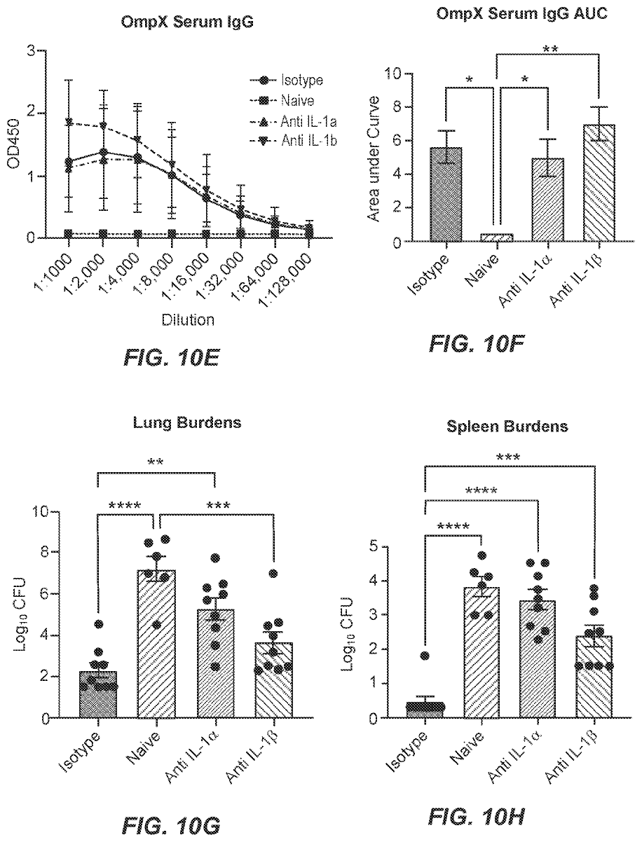

In some embodiments of the invention, the GNB immunogen in the immunogenic compositions is an outer membrane vesicle, or “OMV” of a GNB. GNB are known to produce and release OMVs. In some embodiments, the GNB immunogen is an outer membrane protein, or “Omp,” of a Gram-negative bacterium. Gram-negative bacteria typically produce a number of Omps. K. pneumoniae, for example, produces Omps which are known as OmpX, OmpC, OmpW, and Omplolb. (The sequences of these Omps, each of which is a porin, is set forth in, for example, FIG. 10 of Kolls et al., U.S. Patent Application Publication No. 2022/0160857 A1.) It is expected that other GNB and, in particular, Enterobacteriaceae, comprise Omps that can be used as antigens in combination with IL-1α to provide immunogenic compositions that raise a robust immune response against the organism from which the Omps were derived. It is noted that the human intestinal tract is typically populated with a large population of harmless E. coli, but some strains of E. coli, such as O157:H7, produce potentially lethal toxins. Antigens, such as Omps, for use in immunogenic compositions intended to immunize a subject against infection by a pathogenic strain or serotype of E. coli are preferably selected from antigens that are present in the pathogenic strain or serotype of E. coli against which immunization is intended, but that are absent in harmless strains. In some embodiments, if OMVs are used, they are not from Pseudomonas organisms. In some embodiments, one or more Omps are administered as separate peptides while one or more are in a fusion protein with IL-1α.

Immunogenic compositions of the invention will typically comprise one or more Omps from the Gram-negative bacterium against which the practitioner wishes to raise an immune response, and IL-1α. An immunogenic composition targeted against K. pneumoniae, for example, may comprise OmpX and IL-1α, as in the exemplar composition employed in the studies reported in the Examples, or may comprise OmpX with one or more of OmpC, OmpW, and Omplolb, or of any of OmpC, OmpW, and Omplolb, alone or in combination with one or more of the others, but without OmpX. To avoid “escape” by the target organism, it is anticipated that in some embodiments, the immunogenic compositions will typically comprise more than one of these Omps. One or more of the Omps in an immunogenic composition against a GNB may be in a fusion protein with IL-1α, with one or more additional Omps admixed with the fusion protein, or the composition may contain two or more fusion proteins, each containing a different Omp. In some embodiments, a fusion protein may comprise two or more Ompsut not IL-1α), and the fusion protein can then be admixed with or co-administered with IL-1α.

Fusion proteins are typically expressed as a linear sequence of amino acid residues. In some embodiments, a fusion protein may comprise two or more Omps and IL-1α. In these proteins, one of the two or more Omps will be physically closer in the peptide sequence in the fusion protein to the IL-1α than the other. In some embodiments, the Omp on such a protein may be connected to the IL-1α through a peptide linker, such as a GS linker. In some embodiments, two of the Omps in a fusion protein comprising more than one Omp may be linked to each other through a peptide linker, such as a GS linker. In an embodiment comprising three Omps, or four Omps, each of the Omps may be linked to each other through a peptide linker, such as a GS linker.

As noted, in some embodiments the immunogenic composition may comprise GNB OMVs admixed with IL-1α. “Outer membrane vesicles,” which are sometimes referred to herein as “OMVs,” are “spherical buds of the outer membrane filled with periplasmic content . . . commonly produced by Gram-negative bacteria.” Schwechheimer and Kuehn, Nature Reviews Microbiology, 2015, 13:605-619 (see, abstract). The OMVs produced by those GNB are “spherical, bilayered membrane nanostructures that contain many components found within the parent bacterium.” Kaparakis-Liaskos and Ferrero, Nature Reviews limnunology, 2015, 15:375-387 (see, abstract). OMVs from a number of GNB have been studied for use to immunize subjects against diseases caused by the GNB from which they are derived. See, Kaparakis-Liaskos and Ferrero, supra.

### DEFINITIONS

As used herein, the term “immunogen” refers to a substance that has the ability to evoke an immune response, either by inducing generation of antibodies, by causing a cell-mediated immune response, or by inducing both an antibody and a cell-mediated immune response.

As used herein, “adjuvant” refers to a substance that is capable of enhancing, accelerating, or prolonging an immune response to an immunogen when co-administered with the immunogen.

“Outer membrane vesicles,” which are sometimes referred to herein as “OMVs,” are “spherical buds of the outer membrane filled with periplasmic content . . . commonly produced by Gram-negative bacteria.” Schwechheimer and Kuehn, Nature Reviews Microbiology, 2015, 13:605-619 (see, abstract). Kaparakis-Liaskos and Ferrero state that OMVs are “spherical, bilayered membrane nanostructures that contain many components found within the parent bacterium.” Kaparakis-Liaskos and Ferrero, Nature Reviews Immunology, 2015, 15:375-387 (see, abstract). See also, Kuehn and Kesty, Genes & Dev. 2005. 19: 2645-2655, Kulp and Kuehn, Annual Review of Microbiology, 2010, 64:163-184.

As used herein, “derived from,” with respect to an immunogen, refers to obtaining an immunogenic component of a pathogen or a cancer cell by any of a number of means known in the art, such as by isolation of the immunogen from the native organism or by recombinant expression or synthesis. Immunogens derived from a pathogenic organism may be treated before use to reduce undesired effects. For example, “toxoids” are bacterial toxins which have been treated to suppress or eliminate their ability to act as a toxin, while retaining their ability to induce an immune response against the bacteria from which the toxin originated. The term “derived from” also encompasses structures formed by proteins or peptides that have been recombinantly expressed, such as the virus-like particles that self-assemble from recombinantly expressed capsid proteins of viruses such as human papillomavirus.

As used herein, “co-administration” refers to co-localized administration of two or more agents, such as an immunogen and an adjuvant, to the same subject during a treatment period. The two or more agents may be encompassed in a single formulation and thus be administered simultaneously. Alternatively, the two or more agents may be in separate physical formulations and administered separately to the same spot, or as close as possible to the same spot, in the subject, either sequentially or simultaneously. The term “administered simultaneously” or “simultaneous administration” means that the administration of the first agent and that of a second agent overlap in time with each other, while the term “administered sequentially” or “sequential administration” means that the administration of the first agent and that of a second agent does not overlap in time with each other, but takes place sufficiently close in time that the first agent has not been taken up or metabolized before administration of the second agent so that antigen-presenting cells in the area in which the agents were administered “see” the first agent in conjunction with the second agent.

“Immune response” refers to any detectable response to a particular substance (such as an antigen or immunogen) by the immune system of a host vertebrate animal, including, but not limited to, innate immune responses (e.g., activation of Toll receptor signaling cascade), cell-mediated immune responses (e.g., responses mediated by T cells, such as antigen-specific T cells, and non-specific cells of the immune system), and humoral immune responses (e.g., responses mediated by B cells, such as generation and secretion of antibodies into the plasma, lymph, and/or tissue fluids). Examples of immune responses include an alteration (e.g., increase) in Toll-like receptor activation, lymphokine (e.g., cytokine (e.g., Th1, Th2 or Th17 type cytokines) or chemokine) expression or secretion, macrophage activation, dendritic cell activation, T cell (e.g., CD4+ or CD8+ T cell) activation, NK cell activation, B cell activation (e.g., antibody generation and/or secretion), binding of an immunogen (e.g., antigen (e.g., immunogenic polypeptide)) to an MHC molecule, induction of a cytotoxic T lymphocyte (“CTL”) response, induction of a B cell response (e.g., antibody production), and, expansion (e.g., growth of a population of cells) of cells of the immune system (e.g., T cells and B cells), and increased processing and presentation of antigen by antigen presenting cells. The term “immune response” also encompasses any detectable response to a particular substance (such as an antigen or immunogen) by one or more components of the immune system of a vertebrate animal in vitro.

An “immunological response” to a selected antigen or composition is the development in a subject of a humoral and/or a cellular immune response to molecules present in the composition of interest. For purposes of the present invention, a “humoral immune response” refers to an immune response mediated by antibody molecules, while a “cellular immune response” is one mediated by T-lymphocytes, by other white blood cells, or by both. One important aspect of cellular immunity involves an antigen-specific response by cytolytic T-cells (“CTLs”). CTLs have specificity for peptide antigens that are presented in association with proteins encoded by the major histocompatibility complex (MHCI) and expressed on the surfaces of cells. CTLs help induce and promote the intracellular destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHCII molecules on their surface. A “cellular immune response” also refers to the production of cytokines, chemokines and other such molecules produced by activated T-cells and/or other white blood cells, including those derived from CD4+ and CD8+ T-cells. A composition or vaccine that elicits a cellular immune response may serve to sensitize a vertebrate subject by the presentation of antigen in association with MHC molecules at the cell surface. The cell-mediated immune response is directed at, or near, cells presenting antigen at their surface. In addition, antigen-specific T-lymphocytes can be generated to allow for the future protection of an immunized host. The ability of a particular antigen to stimulate a cell-mediated immunological response may be determined by a number of assays, such as by lymphoproliferation (lymphocyte activation) assays, CTL cytotoxic cell assays, or by assaying for T-lymphocytes specific for the antigen in a sensitized subject. Such assays are well known in the art.

The terms “effective amount” or “pharmaceutically effective amount” of an adjuvant composition and antigen, as provided herein, refer to a nontoxic but sufficient amount of the composition to provide the desired response, such as an immunological response, and optionally, a corresponding therapeutic effect, or in the case of delivery of a therapeutic protein, an amount sufficient to effect treatment of the subject, as defined below. The exact amount required will vary from subject to subject, depending on the species, age, and general condition of the subject, the severity of the condition being treated, and the particular macromolecule of interest, mode of administration, and the like. An appropriate “effective” amount in any individual case may be determined by one of ordinary skill in the art using routine experimentation.

The phrase “pharmaceutically acceptable,” in connection with administration of a substance to a human refers to a substance that is generally safe for human pharmaceutical use. In connection with administration to a non-human animal of a particular species, it refers to a substance that is generally safe and acceptable to a non-human animal of the species in question.

As used herein, the terms “pharmaceutically acceptable carrier” and “pharmaceutically acceptable vehicle” are interchangeable and refer to a fluid vehicle for containing vaccine immunogens that can be injected into a host without adverse effects. Suitable pharmaceutically acceptable carriers known in the art include, but are not limited to, sterile water, saline, glucose, dextrose, or buffered solutions. Carriers may include auxiliary agents including, but not limited to, diluents, stabilizers (i.e., sugars and amino acids), preservatives, wetting agents, emulsifying agents, pH buffering agents, viscosity enhancing additives, colors and the like.

### IL-1α

According to Wikipedia, interleukin-1, or “IL-1,” was discovered in 1972. In 1985, IL-1 was discovered to exist as two separate proteins, now known as IL-1α and IL-1β. See, e.g., March, et al., Nature, 1985, 315(6021):641-7. doi: 10.1038/315641a0. The two separately encoded proteins share only 26% of amino acid homology. See, Afonina et al., Immunity, 2015, 42(6):991-1004. https/doi.org/10.1016/j.immuni.2015.06.003 (“Afonina 2015”). Afonina 2015 states that IL-1α and IL-1β share the same cell surface receptor and “share a practically identical range of biological activities,” but nonetheless show differences in some aspects of immune activation and initiating inflammatory responses. Afonina 2015 speculated that these differences might reflect “differences in localization, expression, and mechanisms of release in different cell types.” The reference explains that IL-1α is “constitutively expressed as a 31-kDa precursor by epithelial cells, endothelial cells, and keratinocytes.” Cleavage of the precursor by any of a number of inflammatory proteases results in a dramatic increase in its bioactivity. Id.

The human gene encoding IL-1α is identified in the database National Center for Biotechnology Information (“NCBI”) as Gene ID 3552. According to the Summary provided by the NCBI under the listing for the gene: “The protein encoded by this gene is a member of the interleukin 1 cytokine family. This cytokine is a pleiotropic cytokine involved in various immune responses, inflammatory processes, and hematopoiesis. This cytokine is produced by monocytes and macrophages as a proprotein, which is proteolytically processed and released in response to cell injury, and thus induces apoptosis. This gene and eight other interleukin 1 family genes form a cytokine gene cluster on chromosome 2. It has been suggested that the polymorphism of these genes is associated with rheumatoid arthritis and Alzheimer's disease.”

### Fusion Proteins and Peptide Linkers

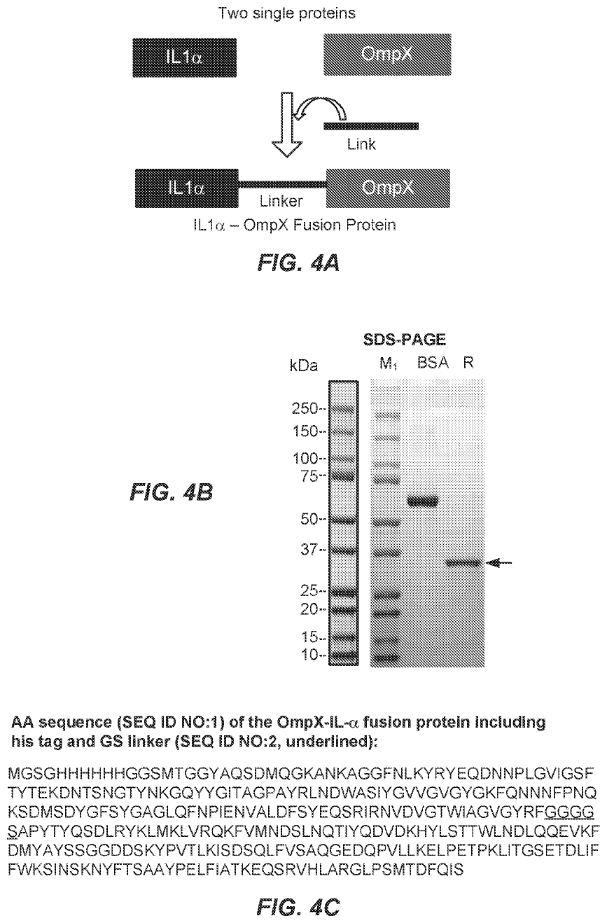

Production of fusion proteins is well known in the art and it is expected that practitioners are well familiar with designing nucleic acid sequences and for methods of expressing such fusion proteins. Similarly, it is expected that practitioners are familiar with the choice of peptide linkers to link together components of a fusion protein. The exemplar OMP-IL-1α fusion proteins used in the studies reported in the Examples used a GS linker, the sequence of which (SEQ ID NO:2) is underlined in FIG. 4C. Fusion proteins are typically used in embodiment in which the GNB, Enterobacteriaceae, or K. pneumoniae immunogen is a protein, such as an Omp, as opposed to, for example, a vesicle.

The peptide is a short sequence of amino acids (3-20, more preferably 3-15, more preferably 3-12, more preferably 4-9, still more preferably 4-8 or 4-7) that serves as bridge between the GNB, Enterobacteriaceae, or K. pneumoniae immunogen and the IL-1α component of a fusion protein, such as an OMP-IL-1α.

A variety of linker peptides are known. For example, Kolls et al., International Publication No. WO 2021/217120, reported the use of such linkers as 1-E-G-R (SEQ ID NO:3), and the thrombin cleavage site, L-V-P-R-G-S(SEQ ID NO:4). The authors of that publication further reported that more than one residue of the same amino acid could be used and that all the residues in the linker could be the same, such as alanines (e.g., A-A-A-A (SEQ ID NO:5) or A-A-A-A-A (SEQ ID NO:6)). The amino acids in the linker are preferably selected from those that are uncharged at physiological pH. If an amino acid that has a charge at physiological pH is used (as in I-E-G-R (SEQ ID NO:3), there is preferably either just one (as in the thrombin cleavage site sequence (SEQ ID NO:4)) or, if two or more are used, there is preferably at least one residue with an opposite charge to keep the overall charge on the linker at 0 or ±1. For example, in the I-E-G-R (SEQ ID NO:3) linker, the acidic residue E is balanced by the basic amino acid R. If two or more charged amino acids are included in the linker, they are preferably separated by at least one amino acid. The amino acids selected for the linker also preferably do not contain aromatic side groups.

In preferred embodiments, the linker is a flexible linker. In some of these embodiments, the flexible linker is a flexible “GS” linker, particularly those described by the formula (GGGGS)ₙ. Flexible GS linkers have been shown to improve the folding and stability of several fusion proteins. The use of peptide linkers in fusion proteins, and the use of GS peptides in particular, is reviewed in, e.g., Chen et al. (Adv Drug Deliv Rev., 2013, 65(10): 1357-1369. doi:10.1016/j.addr.2012.09.039) (“Chen 2013”). Exemplar GS linkers include GGGGS (SEQ ID NO:2), GGGGSGGGGS (SEQ ID NO:7), GGGGSGGGGSGGGGS (SEQ ID NO:8), or GGGGSGGGGSGGGGSGGGGS (SEQ ID NO:9).

Chen 2013 provides an extensive review of linkers that had been used in fusion proteins as of its 2013 publication date. It is expected that the practitioner is familiar with Chen 2013 and with more recent literature concerning the use of linkers in fusion proteins, and is capable of selecting linkers ones suitable for use in the inventive fusion proteins. Any particular linker can be readily tested for its suitability with any particular combination of (a) a GNB, an Enterobacteriaceae, or a K. pneumoniae immunogen, and (b) IL-1α, by substituting the test linker for the GS linker used in the studies reported in the Examples and seeing if the resulting fusion protein has a similar ability to reduce bacterial burden in the same mouse model as the exemplar fusion protein used in the Examples. Linkers that do not reduce the ability of the resulting fusion protein to reduce bacterial burden to the same extent as those in the Examples at similar time points and that do not otherwise cause adverse effects on the animals, are satisfactory.

Diseases for which the Immunogenic Compositions can be Used as Adjuvants

In some embodiments, the immunogenic compositions can prevent illness from, or reduce the severity of, infections by Gram-negative bacteria, or “GNB.” In some embodiments, the GNB are Enterobacteriaceae. In some embodiments, the Enterobacteriaceae is K. pneumoniae.

In general, adjuvants increase the immune response to an immunogen and can be used with immunogens derived from a variety of pathogens. According to the website of the Centers for Disease Control and Prevention (“CDC”), for example, aluminum gels or salts (collectively, generally referred to as “alum”) are used in vaccines against hepatitis A, hepatitis B, diphtheria-tetanus-pertussis (DTaP, Tdap), Haemophilus influenzae type b (Hib), human papillomavirus (HPV) and pneumococcus infection—that is, against infections caused by both viral and bacterial agents. As shown in the studies reported herein, the immune response raised by the exemplar Omp in combination with IL-1α is broader than that of alum and the immunogenic compositions of, for example, OMVs or Omps and IL-1α are therefore expected to be surprisingly superior in raising an immune response to the GNB or Enterobacteriaceae from which the OMVs or Omps were derived compared to immunogenic compositions adjuvanted with alum.

Vaccines for many GNB which can be potentiated by the use of IL-1α are known or under development, as exemplified by vaccines. The immunogen in the vaccine may be, for example, a killed or an inactivated GNB, a live attenuated bacterium, a capsular polysaccharide, alone or conjugated to a carrier protein, such as are used in vaccines directed against Haemophilus influenzae, Neisseria meningitidis, and Salmonella typhi, an OMV (as noted earlier, only GNB release OMVs), or a GNB-derived Omp.

### Formulations, Dosage, and Administration

Formulation and administration of vaccines, and the use of adjuvants is well known, as exemplified by U.S. Pat. Nos. 6,869,607 and 7,371,395. The immunogens used in vaccines traditionally have included killed or attenuated pathogens, such as viruses, bacteria, or protozoans. In some embodiments, a GNB-vaccine or Enterobacteriae-vaccine containing killed or attenuated pathogens may be adjuvanted with IL-1α to enhance its immunogenicity. More recent vaccines contain synthetic or recombinant proteins or peptides. See, e.g., U.S. Pat. Nos. 7,192,595; 6,194,546; 5,962,298; 5,716,620, and 5,437,951. See also, e.g., Schijns and O'Hagan, IMMUNOPOTENTIATORS IN MODERN VACCINES, 2^{nd} Ed., (Academic Press, London, 2017). Recombinant proteins or peptide vaccines are considered as safer than vaccines containing killed or attenuated pathogens, but are often less immunogenic. Embodiments of the invention employing fusion proteins are expected to combine the safety of recombinant proteins over killed or attenuated pathogens, while having better immunogenicity than that seen in recombinant protein or peptide vaccines that do not comprise IL-1α or a comparable adjuvant.

In some embodiments, the inventive immunogenic compositions comprise an immunologically effective amount of the desired GNB or Enterobacteriaceae immunogen and an immunologically effective amount of IL-1α as an adjuvant. Stabilizers, buffers, and other agents known in the art may be added to the vaccine formulation, based on considerations such as how the vaccine composition is going to be stored and the intended route of administration. It is expected that persons of skill in the art are familiar with determining whether any particular vaccine formulation should contain a stabilizer, a buffer, excipients, or other reagents to maximize the shelf-life, effectiveness, or other characteristics of the vaccine. Such vaccine formulations may also be referred to as “pharmaceutical compositions.”

While characteristics of a vaccine formulation may be enhanced by the presence of stabilizers, buffers, or other reagents, the improved immune response to a vaccine formulation comprising a desired GNB, Enterohacteriaceae, or K. pneumoniae immunogen and IL-1α as an adjuvant compared to the same immunogen adjuvanted with another adjuvant is understood to be due to the response by the subject's immune system to the immunogen when the immunogen is presented to the subject's immune system in combination with IL-1α.

By “immunologically effective amount”, it is meant that the administration of that amount to an individual, either in a single dose or as part of a series, is effective in raising an immune response that can ameliorate the symptoms of a disease or condition, or prevent the patient from developing the disease or condition. This amount typically varies depending upon the health and physical condition of the individual to be treated, age, the taxonomic group of individual to be treated (e.g. primate, equine, bovine, porcine, feline, canine, camelid, etc.), the capacity of the individual's immune system to synthesize antibodies or to initiate a cell-mediated immune response, the degree of protection desired, the formulation of the vaccine, the treating doctor's assessment of the medical situation, and other relevant factors. It is expected that the amount will fall in a relatively broad range that can be determined through routine trials. Dosage treatment may be a single dose schedule or a multiple dose schedule (e.g. including booster doses). The vaccine composition may be administered in conjunction with other immunoregulatory agents. Both human and veterinary uses of the inventive compositions and methods are contemplated.

The immunogenic compositions of some embodiments of the invention are preferably administered in effective amounts. An “effective amount” is that amount of a vaccine or immunogenic composition that alone or together with further doses, produces the desired response. In the case of preventing or reducing the severity of a selected infectious disease, the desired response is providing some or complete protection from infection, or amelioration of symptoms, in an individual challenged by an agent that causes the selected infectious disease, compared to an individual who has need received the immunogenic composition.

As persons of skill in the art are aware, the amounts of immunogens and of adjuvants needed to induce an immune response to a typical vaccine are quite small. For example, the Institute for Vaccine Safety states that the DAPTACEL® vaccine contains 10 mcg of pertussis antigen, and 0.33 mg of aluminum phosphate as adjuvant, while the competing INFANRIX@vaccine contains 25 meg of pertussis antigen and 0.625 mg of aluminum hydroxide as adjuvant. It is expected that the dosages of immunogen and of IL-1α as adjuvant will be adjusted based on doses tested in Phase 1 clinical trials. Such testing to adjust dosages is considered routine in the art.

Typically, immunogenic compositions are prepared as injectables, either as liquid solutions or suspensions; solid forms suitable for solution in, or suspension in, liquid vehicles prior to injection may also be prepared. The preparation also may be emulsified for enhanced effect. It is contemplated that compositions comprising a GNB immunogen and IL-1α, either as an admixture or as a fusion protein, may be lyophilized to improve their shelf-life and to reduce or remove the necessity for cold storage. Such lyophilized compositions are typically mixed with a suitable liquid carrier, such as sterile saline, prior to administration to the subject.

To induce the desired mucosal immune response, delivery of the compositions are by intranasal, intrapulmonary, intraperitoneal, subcutaneous, intramuscular, or intracavity administration. To prevent or to reduce occurrence of GNB-related vaginosis, in some embodiments, the compositions may be administered intravaginally. In some preferred embodiments, the compositions are administered by intranasal or pulmonary administration. As noted above, administration may be on a single dose schedule or a multiple dose schedule (e.g. including booster doses).

### EXAMPLES

### Example 1

This Example discusses studies showing that IL-1 signaling is essential for an exemplar vaccine using LTA1 as an adjuvant.

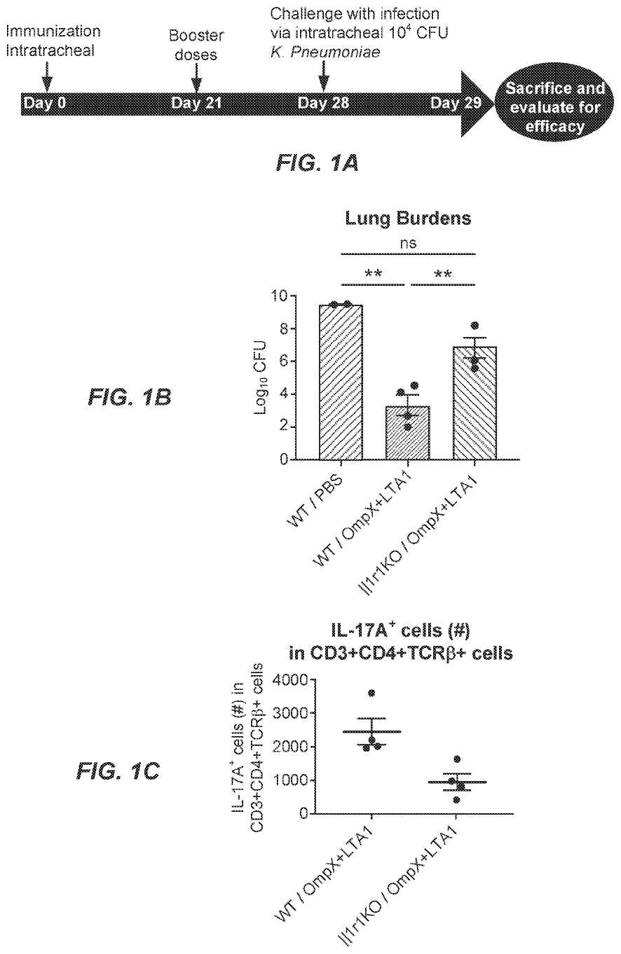

The adjuvanticity of the majority of mucosal adjuvants is due to the ability of the adjuvants to prompt the production of pro-inflammatory cytokines locally and to create an environment in which mucosal immune responses are elicited. IL-1 plays role in enhancing antigen-presenting cell (“APC”) function and also induces production of other cytokines. We studied whether IL-1α signaling is required for the efficacy of an immunogenic composition of K. pneumoniae OmpX+LTA1 using knock-out mice lacking the IL-1 receptor (“IL1r1KO”) and wild type C57BL/6 mice. As shown in FIG. 1, we found that IL1R1 signaling was essential for OmpX+LTA1 vaccine-mediated protection.

### Example 2

This Example discusses a study to determine whether the IL-1 signaling shown in Example 1 to be essential for an exemplar vaccine using LTA1 as an adjuvant was due to IL-1β or IL-1α.

Given prior work that LTA1 can activate the inflammasome, an organelle that facilitates the cleavage of IL-1β to its active from, we presumed that IL-1β was the likely ligand for IL-1R1. To test this, we examined the OmpX+LTA1 vaccine efficacy in wild type mice and mice deficient in caspase-1, a molecule which is essential for the processing and secretion of IL-1β.

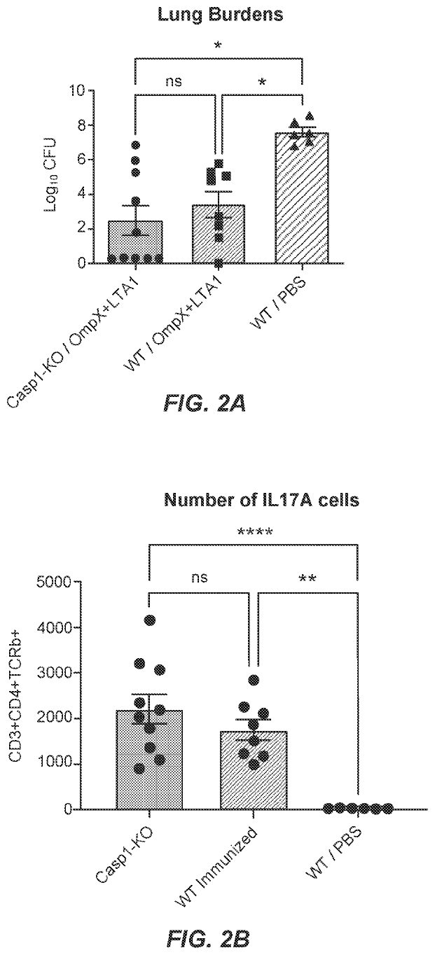

Surprisingly, as shown in FIGS. 2A and 2B, we found vaccine efficacy was maintained in mice deficient in caspase-1 which clearly indicated it is not the IL-1β, but IL-1α, which is playing a critical role in and maintaining vaccine efficacy

### Example 3

This Example discusses a study taken after determining, in the study discussed in Example 2, that IL-1α signaling was essential for efficacy of the exemplar vaccine using LTA1 as an adjuvant.

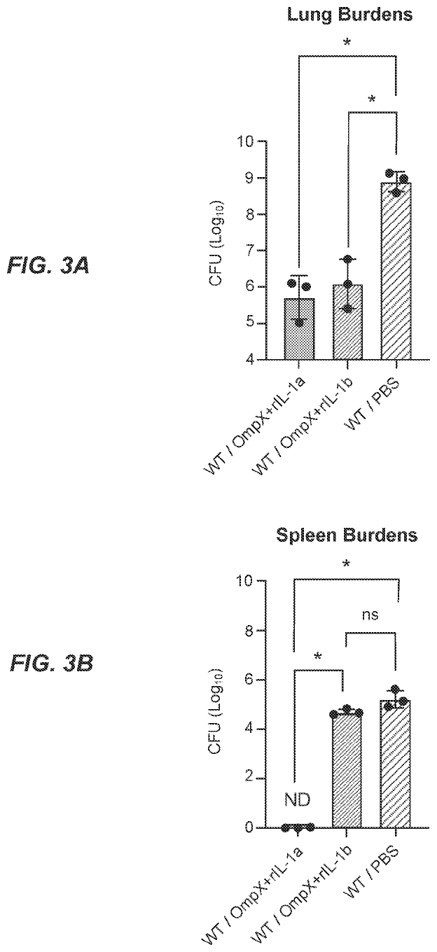

A study was undertaken to see if IL-1α could serve as an adjuvant in place of LTA1. To test this, we conducted intrapulmonary immunization of wild type C57BL/6 mice with 1 μg OmpX+2.5 μg recombinant IL-1α in PBS or 1 μg OmpX+2.5 μg rIL-1β in PBS, or PBS alone (Vehicle control) as control. We were surprised to see that OmpX adjuvanted with rIL-1α showed superior efficacy over OmpX adjuvanted with IL1β in controlling both lung and spleen bacterial burden (FIGS. 3A and 3B).

### Example 4

This Example discusses a study in which a novel fusion protein was constructed.

An exemplar fusion protein was constructed fusing OmpX, marked with a “His tag” (a polyhistidine tag to facilitate purification), to IL-1α through a GS linker. A schematic of the fusion protein is shown in FIG. 4A. FIG. 4B shows a SDS-PAGE analysis of the fusion protein for purity. The band showing the recombinant fusion protein is marked with an arrow. FIG. 4C shows the sequence for the exemplar fusion protein (SEQ ID NO:1), with the GS linker underlined.

### Example 5

This Example discusses a study in which the fusion protein described in Example 4 was tested as a mucosal vaccine.

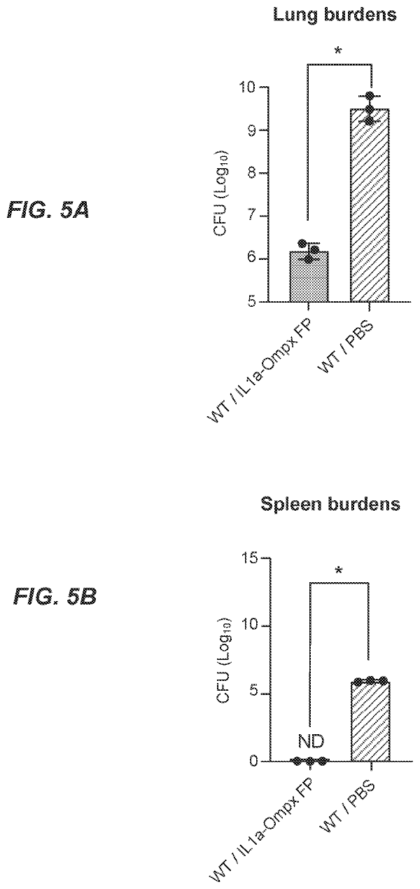

The OmpX-IL-1α fusion protein described in Example 4 was tested in C57BL/6J mice model as described in FIGS. 5A and 5B. As shown in those Figures, mice immunized with the exemplar ILIl-OmpX fusion protein and then challenged with the K1 strain of Klebsiella pneumoniae developed a significantly lower bacterial burden in their lungs and spleens compared to like mice immunized with phosphate-buffered saline and then challenged with the same bacteria.

### Example 6

This Example discusses a study to determine the effects on lung T cells of using LTA1 as an adjuvant compared to using IL1α as an adjuvant.

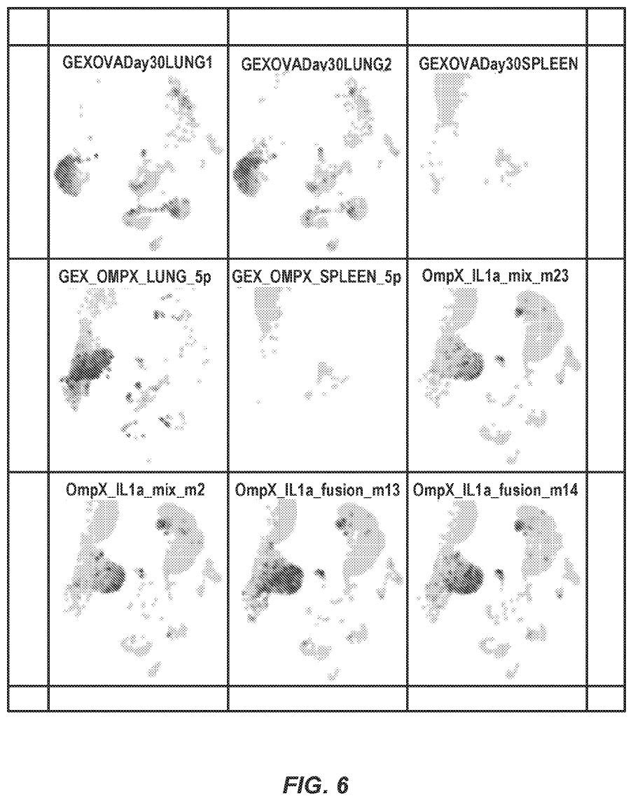

We next compared the transcriptomes of lung CD4+ TRM cells after immunization with Ompx+LTA1, with OmpX admixed with IL1α, or with the exemplar OmpX-IL1α fusion protein described in Example 4. As shown in FIG. 6, UMAP analyses showed that both admixing IL1α or fusing IL1α to OmpX elicited T cells that were highly similar to those elicited by administering OmpX+LTA1 to the animals.

### Example 7

The HIV epidemic clearly demonstrated the critical roles CD4+ T cells play in host resistance to pneumonia. As such, development of vaccines that elicit these cells at mucosal sites of infection is of great interest. Prior work from our group has shown that complex antigen exposure, such as immunization with heat-killed K. pneumoniae, results in the generation of protective lung Th17 TRM cells in mice. (See, Chen, et al., Immunity. 2011; 35(6):997-1009.) We further demonstrated a mucosal subunit vaccine consisting of the bacterial outer membrane protein OmpX from K. pneumoniae and the adjuvant composed of the A1 domain of E. coli heat labile toxin (LTA1) also induces a robust enrichment of lung Th17 TRM cells that protect against pulmonary challenge with K. pneumoniae. (See, Iwanaga, et al., Sci Immunol, 2021, 6(6):eabf1198 (“Iwanaga 2021”)). Given that OmpX is a highly conserved bacterial protein, these elicited T cells were able to recognize other Enterobacteriaceae family members that also encode OmpX. Development of such broadly acting vaccines offers an advantage over traditional vaccines that only elicit humoral responses. However, the relative roles of upstream cytokines in directing the development of vaccine-elicited Th17 TRM cells from naïve T cell progenitors remain unknown.

Studies examining the effects of LTA1 in THP-1 cells found LTA1 activates the NLRP3 (NOD-, LRR- and pyrin domain-containing protein 3) inflammasome, leading to cleavage of pro-caspase-1 into caspase-1 and subsequent secretion of IL-1β. Importantly, IL-1R1 signaling has been linked to the activation and expansion of Th17 cells. See, e.g., Chung, et al., Immunity, 2009, 30(4):576-87. In particular, IL-1β promotes Th17 cell differentiation in humans and mice and enhances memory T cell activation and proliferation. See, e.g., Acosta-Rodriguez, et al., Nat Immunol., 2007, 8(9):942-9. A similar role for IL-1α in survival and expansion of memory CD4+ T cells has also been suggested. However, whether IL-1 signaling is critical for vaccine-mediated Th17 cell generation is unknown. A greater understanding of the mechanisms through which naïve T cells develop into Th17 TRM cells upon immunization would enable development of improved mucosal vaccines.

The studies reported below demonstrate a role for IL-1-related cytokines in Th17 TRM cell generation following immunization with OmpX+LTA1. In addition, they assess the use of these cytokines as mucosal adjuvants in a vaccine against an exemplar Gram-negative bacterium, K. pneumoniae.

### Example 8

This Example sets forth materials and methods used in studies discussed below.

### Mice:

For all in vivo studies, male mice aged 7-10 weeks on a C57Bl/6J background were used. Wildtype C57Bl/6 mice used in the study were either bred in house or purchased from the Jackson Laboratory. Transgenic mouse strains Il1r1^{-/-} (Strain #:003245), Casp1^{-/-} (Strain #:032662), Casp1/4^{-/-} (Strain #:016621), and Il1a^{Δ559,1} (Strain #: 067031-JAX) were purchased from the Jackson Laboratory. All mice were housed in specific pathogen free conditions at Tulane University School of Medicine and provided food and water ad libitum. Experimental procedures were conducted in accordance with protocols approved by Tulane University's Institutional Animal Care and Use Committee.

### Antigen and Adjuvant Preparation and Vaccination Strategy:

The vaccine antigen, recombinant OmpX, was produced and purified using an Escherichia coli expression system as described in Iwanaga 2021. The adjuvant LTA1 was isolated and purified using a methodology previously described in Valli, et al., Sci Rep., 2019, 9(1):15128. Recombinant IL-1α and IL-1β used as adjuvants were purchased from R&D Systems (Cat #400-ML-005/CF and 401-ML-010/CF). Mice were vaccinated following the timeline and dosing as described in Iwanaga 2021. In brief, mice were vaccinated following a prime/boost strategy, with the boost occurring 21 days following the prime. Each vaccination dose consisted of 1 μg OmpX mixed with 10 μg LTA1 in a 50 μL volume of PBS. For our studies using rIL-1α and rIL-1β as adjuvants, 1 μg of OmpX was mixed with 2.5 μg of each respective cytokine in a 50 μL volume of PBS. Each vaccine was administered by direct instillation into the lungs via oropharyngeal aspiration following anesthetization with isoflurane, as described in Nielsen, et al., J Vis Exp, 2018, (136):57672; doi: 10.3791/57672. For all challenge studies, mice were challenged 7-10 days following the vaccine boost.

Oropharyngeal Infection with K. pneumoniae and Enumeration of Bacterial Burdens:

K. pneumoniae-396 (K1) was grown overnight in a 3 mL culture of Luria broth (LB) Miller (VWR) or tryptic soy broth (Difco) at 37 °C with shaking at 233 rpm. Overnight cultures were then subcultured with a 1:100 dilution into 30 mL of the same media. This subculture was grown in the same conditions for 2.5 hours to achieve early logarithmic growth phase. The concentration of K. pneumoniae was determined by reading the optical density at 600 nm. The bacteria were then pelleted via centrifugation at ~5,000 g for 8 minutes and washed 2× with sterile PBS. Bacteria were resuspended in sterile PBS to the desired concentration. To confirm the dose of bacteria, the inoculum was serially diluted in PBS and spot plated on LB agar plates to count CFUs. For infection, mice were anesthetized by isoflurane inhalation and 1×10⁴ CFU of K1 was directly instilled into the lungs via oropharyngeal aspiration in a 50 μL volume. To enumerate bacterial burdens, mice were euthanized via CO₂ asphyxiation at 24 hours post infection. The right lung and spleen were collected and placed in their own tubes of 1 mL of sterile PBS on ice until further processing. The organs were subsequently homogenized using a Multi-Gen 7XL handheld tissue homogenizer (Proscientific). Homogenized tissue was serially diluted in sterile PBS and spot plated on LB agar plates for CFU counts.

### Generation of Single Cell Suspension for Flow Cytometry and ELISpot:

The left lung of euthanized mice was collected in 700 μL sterile PBS and kept on ice for further processing. The PBS was decanted, and the tissue was minced manually with dissection scissors. Minced tissue was resuspending in 2 mL IMDM (Gibco) containing 2 mg/mL collagenase (Sigma-Aldrich) and 80 U/mL DNasel (Sigma-Aldrich) and incubated at 37 °C with shaking at 233 rpm for 1 hour. Digested tissue was passed through a 70 μm cell strainer (Fisher) and red blood cells were removed using ACK lysis buffer (Gibco). Isolated cells were resuspended in 1 mL IMDM containing 10% FBS (Hyclone) and counted on a Cellometer for downstream applications.

### Intracellular Cytokine Staining and Flow Cytometry:

Single cell suspensions were made of lungs collected from vaccinated and infected mice and 1×10⁶ cells from each sample were added to the wells of a 96-well round bottom plate. Cells were stimulated with 50 ng/mL of Phorbol 12-myristate 13-acetate (Sigma-Aldrich) and 750 ng/mL ionomycin (Sigma-Aldrich) for 5 hours at 37 °C. At 1 hour of stimulation, 1 mg/mL GolgiStop (BD Bioscience) was added to prevent the secretion of cytokines. Cells were washed with FACS buffer (1×PBS with 0.5% BSA) and fixed and permeabilized with cytofix/cytoperm (BD Biosciences) for 20 minutes. Cells were washed with 1× Perm/wash buffer (BD Biosciences), Fc receptor blocked, and stained for 30 minutes for desired cell markers. Cells were washed 3×, resuspended in FACS buffer, and analyzed using a Cytek Aurora spectral flow cytometer. For each sample, 5×10⁴ events were recorded and all analysis was conducted using FlowJo software version 9 (Tree Star). Antibodies used for blocking and staining are as follows: Rat Anti-Mouse CD16/CD32 Fc Block (clone 2.4G2, BD Biosciences), PE-Cy7 Rat Anti-Mouse CD4 (clone RM4-5, BD Biosciences), APC rat anti-mouse CD3e (clone 17A2, Biolegend), PE-Cy5 hamster anti-mouse TCRβ (clone H57-597, BD Biosciences), FITC rat anti-mouse IL-17A (clone TC11-18H10.1, Biolegend), Brilliant Violet 421 rat anti-mouse IFNγ (clone XMG1.2, Biolegend), PE rat anti-mouse B220 (clone RA3-6B2, Biolegend), eFluor 450 rat anti-mouse CD19 (1D3, Invitrogen).

### Antibody Neutralization of IL-1a and IL-1p:

Wild type male C57Bl/6J mice aged 8 weeks were vaccinated with OmpX+LTA1 as described above. At 1 day prior to boost, mice were with treated with 200 μg of anti-IL-1α, anti-IL-1β, or anti-IL-1α+ anti-IL-1β neutralizing antibodies or isotype control via intraperitoneal injection in a 200 μL volume. One group of naïve mice were used as an additional control. Vaccinated and antibody treated mice were challenged and euthanized following the vaccination timeline described above. All antibodies were diluted in sterile PBS prior to injection. Neutralizing antibodies were purchased from Bio X cell: anti-IL-1α (BE0243), anti-IL-1β (BE0246), and isotype control (BE0091).

### In Vivo Inflammatory Cytokine Analysis:

Mice were vaccinated following the prime/boost schedule described above with one group receiving OmpX+LTA1 and the other sterile PBS. At 24 hours following the boost, all mice were euthanized and serum, bronchial alveolar lavage fluid (BALF), and supernatant from whole lung homogenate were collected for cytokine analysis. In brief, blood was collected via cardiac puncture and spun at 2,500 rpm on a benchtop microcentrifuge for 20 minutes to collect serum. For BALF, cOmplete™ ULTRA tablets, mini, EASYpack Protease Inhibitor cocktail (Roche, #5892970001) was prepared in PBS following manufacturer's instructions. This solution was instilled into the lungs of mice intratracheally at a 1 mL volume for 3 washes and kept on ice. Whole lungs were then collected in 1 mL of the protease cocktail, homogenized with the handheld tissue homogenizer as described, pelleted at 2,500 rpm for 15 minutes at 4 °C, and the supernatant was collected. Inflammatory cytokines were quantified from each sample using a LEGENDPlex™ mouse inflammation panel (BioLegend, #740150) following manufacturer's instructions. Samples were processed using a Cytek Aurora spectral flow cytometer and analyzed using the LEGENDplex™ Data Analysis Software Suite (Qognit).

### ELISA:

ELISAs were performed to evaluate serum TgG and lung IgA titers following methods set forth in Iwanaga 2021. Serum was collected as described above. Lung IgA was collected in BALF consisting of 1 mL sterile PBS and 3 washes of the lungs. 96 well plates were coated overnight with 0.1 μg heat killed K2 strain K. pneumoniae or rOmpX in 100 μL per well. Coated plates were washed with washing buffer (0.05% Tween 20 in PBS) and blocked for 2 hours with blocking buffer (1% bovine serum albumin and 0.1% Tween 20 in PBS). Following blocking, plates were washed 3× and serially diluted serum or BALF was added. Plates were left to incubate at room temperature for 2 hours and washed 5×. Bacterial specific antibodies were detected using 1:4000 diluted goat anti-mouse IgG or IgA conjugated with horseradish peroxidase (Southern Biotech, Cat #1036-05 and 2050-05) and incubated for 1 hour at room temperature. Plates were washed 5× and 3,3′,5,5′-tetramethyl-benzidine peroxidase substrate (TMB, ThermoFisher, #N301) was added to each well. Absorbance was read at 450 nm on a 96 well plate reader (Biotek). For the quantification of IL-1α, we used the Mouse IL-1alpha ELISA MAX™ Deluxe kit from BioLegend following the manufacturer's recommendations (Cat #433404)

### ELISpot:

ELISpot was used in some experiments for the quantification of IL-17A producing cells in the lungs of immunized mice. To do this, Millipore MultiScreen-IP plates (Millipore Sigma, #MAIPS4510) were activated with 50 μL/well freshly prepared 70% ethanol for two minutes. Plates were washed 4× with PBS and coated with 2.5 μg/mL anti mouse IL-17A antibody (R&D, #MAB721-100) in PBS at 4 °C overnight. Plates were washed 4× with wash buffer (1×PBS with 0.05% Tween 20) and incubated with complete IMDM (IMDM with 10% BSA) for 2 hours at 37 °C. Following incubation, IMDM was removed and 1×10⁵ isolated lung cells and 2 μg/mL OmpX were added in triplicate to a total volume of 100 μL per well. Cells were then incubated at 37 °C with 5% CO₂ for 18 hours. Following incubation, plates were washed 4× with wash buffer and biotinylated anti-mouse IL-17A antibody was added in assay buffer (1×PBS, 0.05% Tween 20, 0.5% BSA) at a concentration of 0.8 μg/mL in a volume of 100 μL per well. Plates were incubated for 2 hours with gentle shaking at room temperature. Plates were washed 4× and incubated with 1:2000 diluted streptavidin-alkaline phosphatase (R&D, #AR001) for 45 minutes with gentle shaking at room temperature. Plates were washed once more and spots were developed using BCIP/NBT substrate solution (Sigma-Aldrich, #B5655) for 15 minutes. After development, plates were read on a CTL ImmunoSpot® S4 and analyzed with ImmunoSpot® Software (Cellular Technology Ltd) for the quantification of spot forming units (SFUs).

### Single Cell RNA Sequencing:

Single cell RNA sequencing was published previously in Iwanaga 2021. Essentially, 1×10⁶ cells were collected as whole-lung or whole-spleen single-cell populations. Cells were subjected to enrichment by using a CD4 positive selection kit (catalog no. 130-104-454, Miltenyi Biotec) and treated with 100 μl of TrypLE for 1 min to dissociate single cells from small aggregates or clusters. Cell numbers and viability were validated by Cellometer Auto 2000 (Nexcelom Bioscience) before preparation of scRNA-seq library. For 10× single-cell 3′ RNA-seq assay, 5000 live cells per sample were targeted by using 10× scRNA-seq technology provided by 10× Genomics Inc. (Pleasanton, CA). Briefly, viable single-cell suspensions were partitioned into nanoliter-scale Gel Beads-In-Emulsion (“GEMs”). Full-length barcoded complementary DNAs (cDNAs) were then generated and amplified by PCR to obtain sufficient mass for library construction. After enzymatic fragmentation, end-repair, A-tailing, and adaptor ligation, single-cell 3 libraries comprising standard Illumina P5 and P7 paired-end constructs were generated. Library quality controls were performed by using an Agilent High Sensitive DNA kit with Agilent 2100 Bioanalyzer and quantified with a Qubit 2.0 fluorometer. Pooled libraries at a final concentration of 1.8 pM were sequenced with paired-end single index configuration by Illumina NextSeq 550. Cell Ranger version 2.1.1 (10× Genomics) was used to process raw sequencing data and Loupe Cell Browser (10× Genomics) to obtain differentially expressed genes between specified cell clusters. In addition, Seurat suite version 2.2.1 (Butler, et al., Nat Biotech, 2018, 36(5):411-20) was used for quality control and downstream analysis. Filtering was performed to remove multiplets and broken cells. Also, uninteresting sources of variation were regressed out. Variable genes were determined by iterative selection based on the dispersion versus average expression of the gene. For clustering, principal components analysis was performed for dimension reduction. The top ten principal components were selected by using a permutation-based test implemented in Seurat and passed to t-SNE for clustering visualization. Gene Expression Omnibus accession number is GSE178385.

### Statistical Analysis:

All statistical analysis was conducted using Graphpad Prism (version 9). For analysis comparing two groups, a Student's T test was used. For comparisons of 3 or more groups, we used one-way analysis of variance (ANOVA) with Tukey's post hoc analysis after applying the Bonferroni correction for multiple comparisons. For all analyses involving bacterial burdens, we performed a log transformation on the data and performed ANOVA on transformed data as described above. P-values are annotated as follows: *P≤0.05, **P≤0.01, ***P≤0.001, ****P≤0.0001.

### Study Approval:

Experimental procedures were conducted in accordance with protocols approved by Tulane University's Institutional Animal Care and Use Committee.

### Example 9

This Example discusses the results of studies performed using the materials and methods discussed in the previous Example.

Mucosal Immunization with OnpX+LTA1 Induces IL-1 Signaling In Vivo.

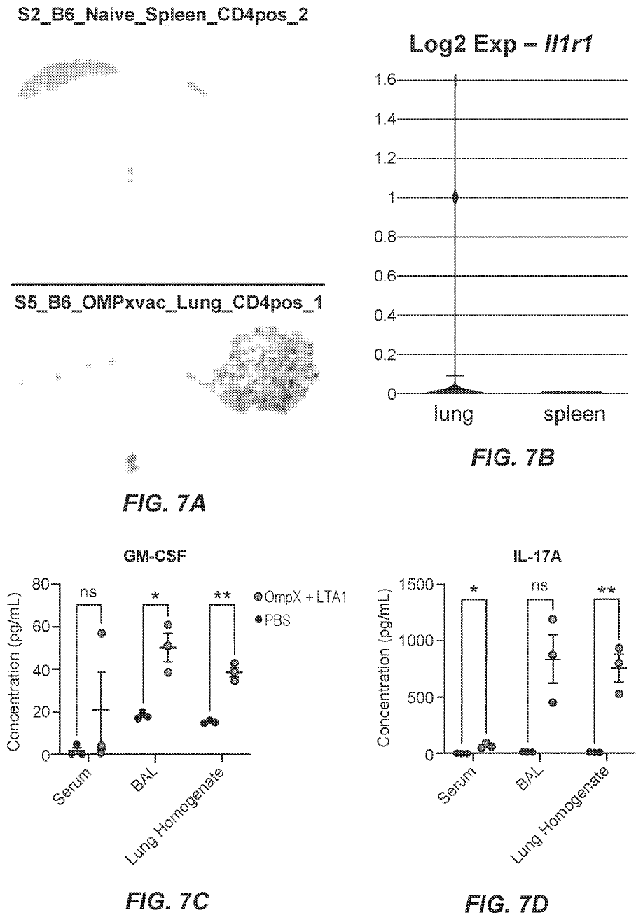

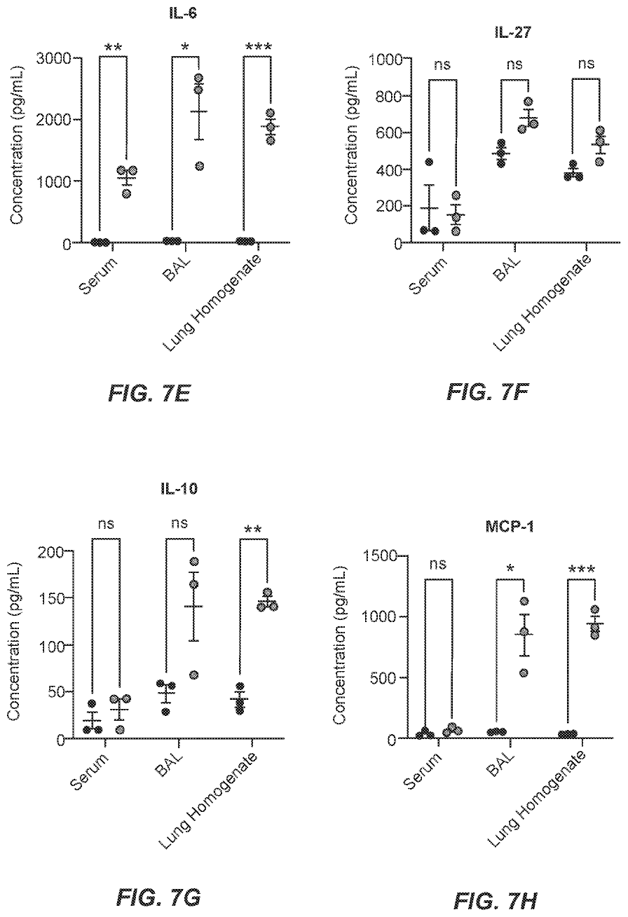

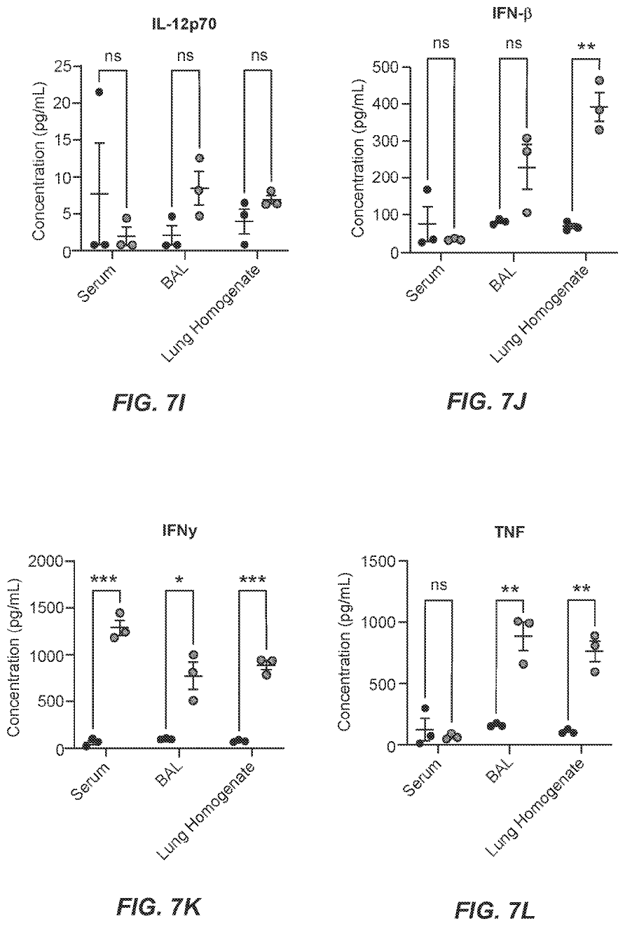

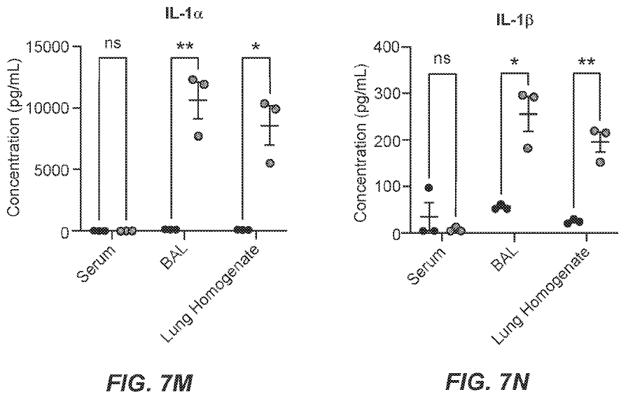

To identify upstream signaling pathways that could play a role in formation of Th17 TRM cells upon vaccination, we examined our existing sc-RNA-seq data comparing CD4+ T cells isolated from the lungs of mice immunized with OmpX+LTA1 and naïve CD4+ T cells from spleens of unimmunized mice. These groups displayed distinct clustering in Uniform Manifold Approximation and Projection (UMAP) plots, suggesting a shift in gene expression of CD4+ T cells following immunization (FIG. 7A). Analysis of these clusters for gene expression differences indicated upregulation of Il1r1 transcript levels in the lung resident CD4+ T cells, while there was no detectable Il1r1 in the CD4+ T cells from naïve mice. (FIG. 7B).

To determine whether vaccination impacted IL-1-related signaling in vivo, C57Bl/6 mice were immunized twice, three weeks apart with oropharyngeal OmpX+LTA1 or administered PBS as described in Iwanaga 2021. One day following the boost, mice were euthanized and the presence of inflammatory cytokines was assessed in the serum, lung tissue homogenate, and BALF of vaccinated mice and PBS administered controls. As expected, vaccinated mice had significantly higher expression of proinflammatory cytokines than their PBS counterparts, particularly in the lungs as measured in the lung homogenate and BALF samples. (FIGS. 7C-N). For recruitment and expansion of innate immune cells, we observed greater expression of GM-CSF and MCP-1 in the lungs (FIGS. 7C and 7H). As demonstrated previously for LTA1 in Twanaga 2021, we saw a significant increase in IL-17A in the lung homogenate and BALF, and a small but significant increase in the serum (FIG. 7D). Additionally, we found vaccination with OmpX+LTA1 led to an increase of the proinflammatory cytokines IL-6, IFNγ, and TNF in the lung homogenate and BALF, with IL-6 and IFNγ also being enhanced systemically as demonstrated by increased levels of these cytokines in the serum (FIGS. 7E, 7K, and 7L). Importantly, we found that immunized mice had significantly elevated amounts of both IL-1α and IL-1β in the lungs with the amounts of IL-1α particularly striking (FIGS. 7M and 7N). All together, these data suggest vaccination with OmpX+LTA1 induces IL-1 signaling in our vaccine model.

### IL-1 Signaling Promotes Formation of Vaccine-Elicited Mucosal Th17 Cells.

After verifying that the exemplar vaccine promotes the expression of IL-1 cytokines in the respiratory tract in vivo, we next investigated whether IL-1 signaling was necessary for generating enriched adaptive immune cell populations and protective immunity. To do this, we utilized Il1r1 global knockout mice (Il1r1^{-/-}). These mice are deficient in the receptor IL-1R1, which both IL-1β and IL-1α can bind to and signal through via MyD88 and subsequently NFκB. As such, Il1r1^{-/-} mice are unable to respond to IL-1β or IL-1α. Both wildtype and Il1r1/mice were immunized with either OmpX+LTA1 or administered PBS as above to determine the effects of IL-1 signaling on vaccine induced immunity. One week following the final boost all mice were challenged with 1×10⁴ CFU K. pneumoniae and euthanized 24 hours after challenge to assess immune populations and bacterial burdens.

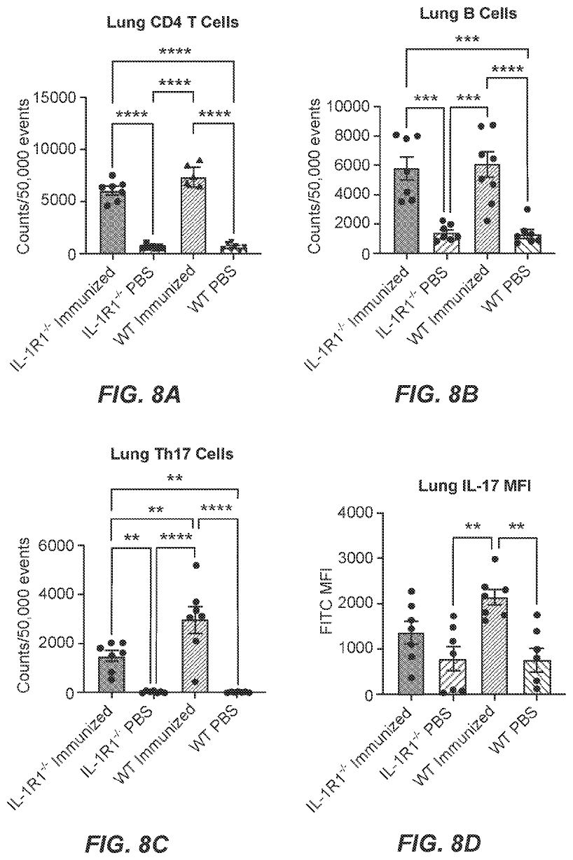

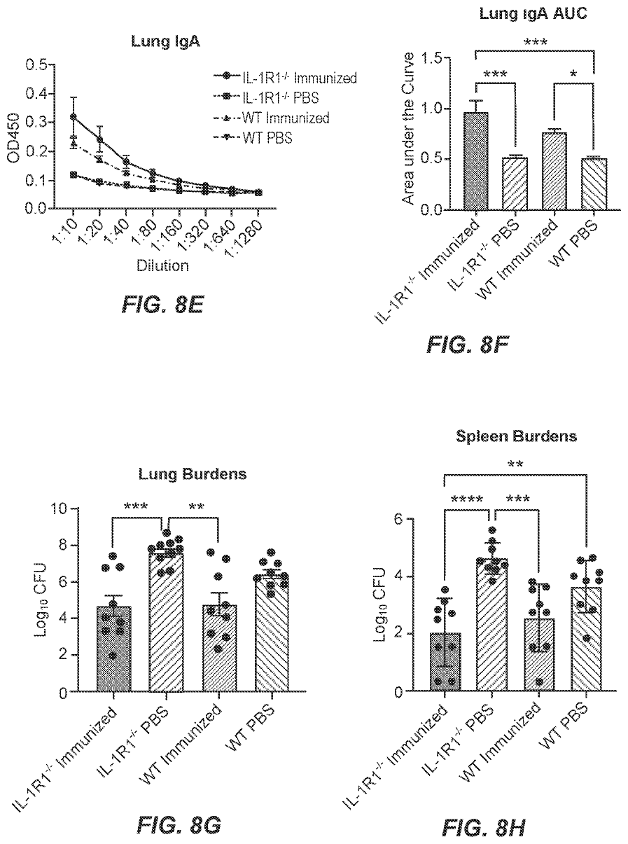

To evaluate adaptive immune populations, we quantified B cells, CD4+ T cells, and Th17 cells in the lungs of all animals using flow cytometry. Adaptive immune cells were identified first by using forward and side scatter to gate for lymphocytes and single cells. From there cells were gated as CD3+ or CD3−. CD3− cells were classified as B cells if they were positive for CD19 and B220. CD3+ cells that were positive for CD4 and TCR-β were identified as CD4 T cells. From this population, Th17 cells were identified based on positive staining for IL-17A. Examination of CD4+ T cell and B cell populations in the lungs of immunized and naïve mice revealed vaccination increased the abundance of these cells in both the wildtype and Il1r1^{-/-} mice compared to PBS-treated controls, though there was no difference in number of CD4+ T cells between the immunized Il1r1^{-/-} mice and their wildtype counterparts (FIGS. 8A-B). Given these data, it appears that IL-1 signaling does not have an impact on the enrichment of total adaptive immune populations in this exemplar vaccine model. Interestingly, though vaccinated Il1r1^{-/-} and wildtype mice had comparable levels of total lung CD4+ T cells, immunized Il1r1^{-/-} had a significantly smaller population of Th17 cells (FIG. 8C). Indeed, the knockout mice had roughly half as many Th17 cells and a trend toward reduced IL-17A median fluorescent intensity (MFI) versus the wildtype mice (FIG. 8E). Of note, PBS treated wildtype and Il1r1^{-/-} mice had similar populations of B cells, CD4+ T cell, and Th17 cells in the lungs (FIGS. 8A-DE), suggesting knockout of IL-1 signaling does not impact these populations in naïve mice. We next evaluated presence of K. pneumoniae specific IgA in lung homogenates. Both immunized wildtype and Il1r1^{-/-} mice had levels of lung IgA that were significantly higher compared to the respective PBS administered groups, though no differences were seen between vaccinated groups (FIGS. 8E-F). Thus, observed immunological differences between the two immunized groups appear to be restricted to Th17 cell populations. Since we have previously demonstrated that Th17 cells are necessary for vaccine mediated protection against pulmonary challenge with K. pneumoniae (see, Iwanaga 2021), we next assessed bacterial burdens in the lungs and spleens from each group 24 hours post-challenge with K. pneumoniae. Surprisingly, though the immunized Il1r1^{-/-} mice had significantly fewer Th17 cells than the wildtype mice, both groups were protected from challenge as demonstrated by bacterial burdens (FIGS. 8G-H). There was no significant difference between the immunized groups and both groups had significantly reduced burdens from their PBS administered counterparts. Taken together, it appears that IL-1 signaling is important but not the only requirement for the expansion of vaccine-induced Th17 cells. While mice without IL-1 signaling had reduced populations of Th17 cells, these cell numbers appeared to be sufficient in conferring protection in our vaccination and challenge model.

### Cleaved IL-1β is Dispensable for Th17 Cell Expansion and Vaccine Mediated Protection.

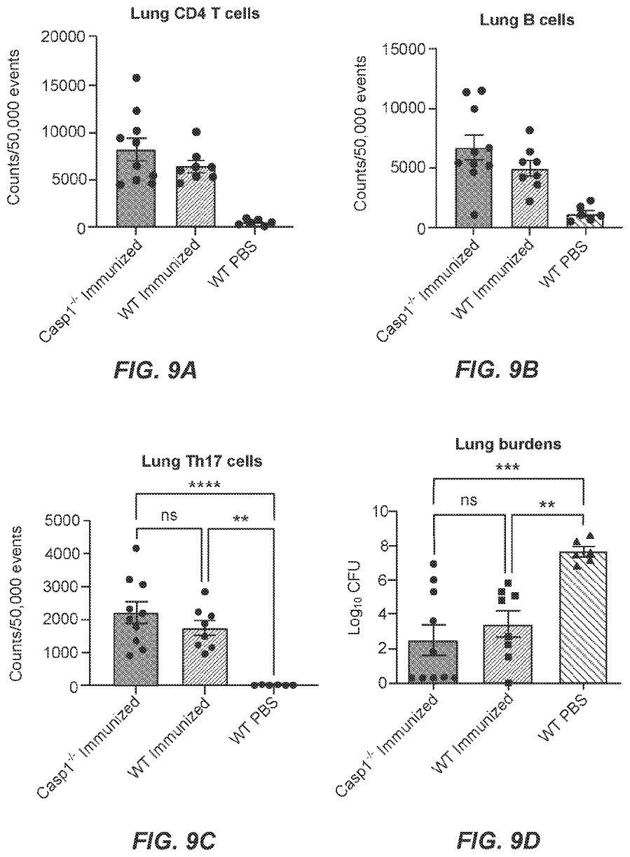

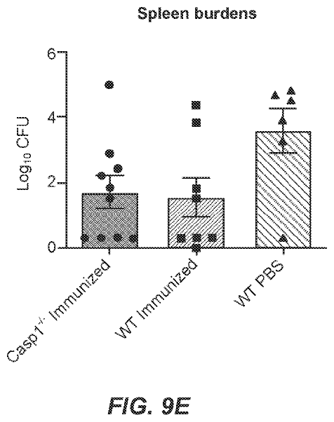

To further investigate how IL-1 signaling influences the expansion of Th17 cells in our vaccination model, we utilized mice deficient in caspase-1 (Casp1^{-/-}). Caspase-1, which is cleaved and activated by the inflammasome complex, proteolytically cleaves cytosolic pro-IL-1β into its active form, IL-1β. Without caspase-1, Casp1^{-/-} mice are unable to generate active IL-1β. Casp1^{-/-} and wildtype mice were vaccinated with OmpX+LTA1 and subsequently challenged as described using a group of wildtype mice administered PBS as a control. Similar to observations in our Il1r1^{-/-} mice, we found that while vaccination increased CD4+ T cells and B cells in both Casp1^{-/-} mice and wildtype mice compared to the PBS-treated control, there was no difference in the number of either cell population between the vaccinated groups (FIGS. 9A-B). These data suggest that the vaccine generated an adaptive immune response in both groups of vaccinated mice independent of active IL-1β. To our surprise, when we compared the number of lung Th17 in the vaccinated wildtype and Casp1^{-/-} groups, we found that the absence of caspase-1 had no impact on the number of Th17 cells (FIG. 9C). Indeed, both immunized Casp1^{-/-} and wildtype mice had comparable numbers of Th17 cells in the lungs. To determine whether absence of caspase-1 affected vaccine efficacy, we challenged mice with K. pneumoniae and evaluated bacterial burdens in the lungs and spleens 24 hours post-infection. Evaluation of bacterial burdens in the lungs revealed both immunized wildtype and Casp1^{-/-} mice were protected from challenge and had on average 6 logs fewer CFU than the PBS control mice (FIG. 9D). In addition, there was no difference in bacteria burdens between the two vaccinated groups. Evaluation of spleen burdens determined that both vaccinated groups trended toward having fewer CFUs in the spleen compared to PBS control mice, indicative of less dissemination, though differences were not statistically significance (FIG. 9E). These data demonstrate that cleaved IL-1β is not required for the generation of lung Th17 cells or vaccine-mediated protection in our model.

IL-1α Enhances Th17 Cell Generation and Promotes Protection Following Vaccination with OmpX+LTA1.

While IL-1R1 signaling enhanced production of vaccine-elicited Th17 cells in the lungs, we were surprised IL-1β was dispensable in this process. Since both IL-1α and IL-1β can signal through IL-1R1, we next investigated whether active IL-1α could be driving Th17 cell enrichment. We investigated the role of IL-1α role using caspase-1 and caspase-4 double knockout mice (Casp1/4^{-/-} mice). These mice are deficient in IL-1β and IL-18 and have a significant reduction in secreted IL-1α, though they are not IL-1α deficient (Kuida, et al., Science, 1995, 267(5206):2000-3). To determine whether active IL-1α was necessary for generation of vaccine-mediated Th17 cells, Casp1/4^{-/-} and wildtype mice were immunized with OmpX+LTA1 using PBS as a control. Vaccination resulted in enrichment of lung CD4+ T cells, B cells, and Th17 cells in both Casp1/4^{-/-} and wildtype mice compared to their respective PBS treated controls. To our surprise, there was no difference in these cell populations between immunized Casp1/4^{-/-} and wildtype mice. Challenge of immunized mice with K. pneumoniae resulted in near identical lung burdens between the vaccinated wildtype and Casp1/4^{-/-} mice 24 hours post-infection. Interestingly, immunized Casp1/4^{-/-} mice fared better than immunized wildtype mice against bacterial dissemination as demonstrated by spleen burdens 24 hours post-infection. Though both groups had significantly lower burdens than their respective PBS controls, we were unable to recover any bacteria from the spleens of immunized Casp1/4^{-/-} mice, while immunized wildtype mice had an average of ~102 CFUs in the spleen. While reduction of IL-1α had no effect on Th17 cell enrichment in this model, it is possible levels of active IL-1α may still be sufficient in Casp1/4^{-/-} mice for promoting a Th17 response upon vaccination.

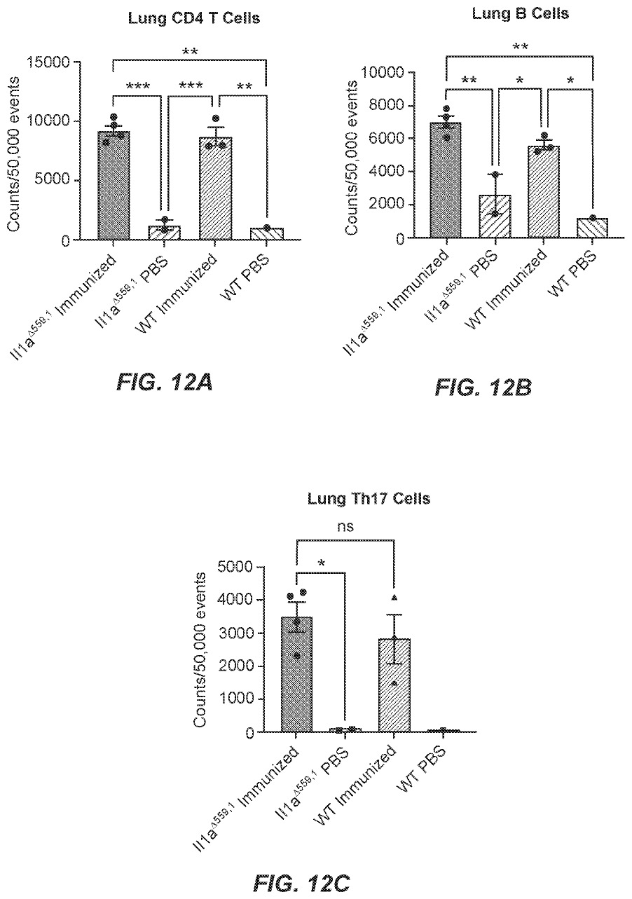

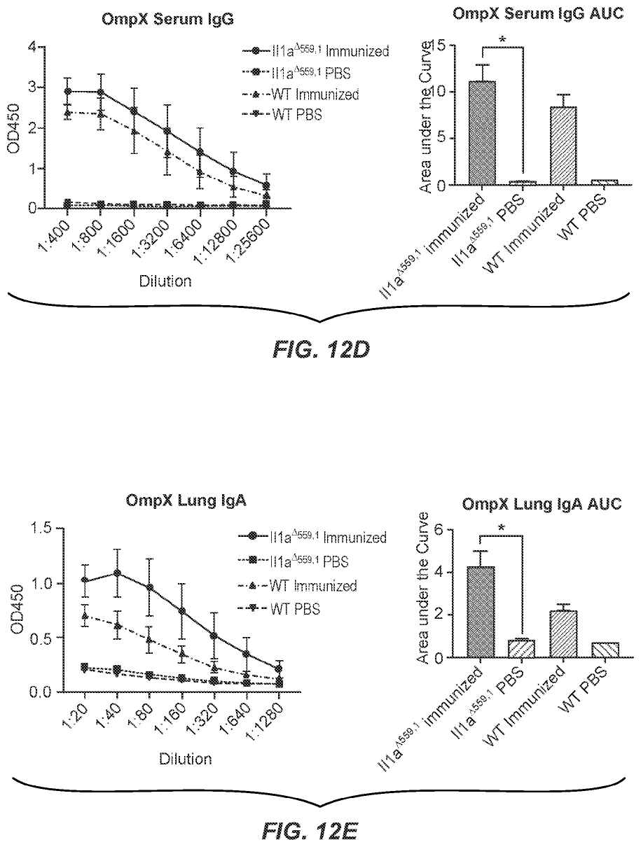

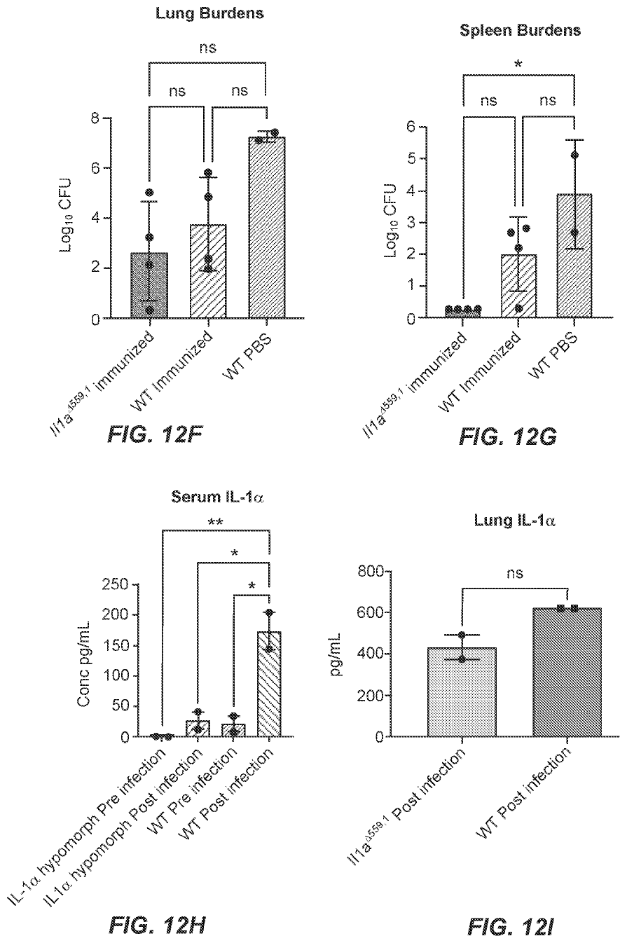

We next used another mouse model deficient in IL-1α(Il1a^{Δ559,1} mice) to determine the role of this cytokine in eliciting Th17 cells upon vaccination. These mice were generated using CRISPR/Cas9 endonuclease-mediated gene editing and carry a 559-nucleotide deletion in Exon 3 of the Il1a gene. As such, Il1a^{Δ559,1} mice have decreased secretory IL-1α levels compared to wildtype mice following 24 h or 48 h stimulation with LPS. Wildtype and Il1a^{Δ559,1} mice were immunized and challenged as above and the immune response and protective efficacy of the vaccine were assessed. Similar to our observations in Casp1^{-/-} and Casp1/4^{-/-} mice, we found that immunized Il1a^{Δ559,1} mice had similar numbers of CD4+ T cells, B cells, and Th17 cells in the lungs compared to immunized wildtype mice (FIGS. 12A-C). Both vaccinated Il1a^{Δ559,1} and wildtype mice had significantly higher numbers of these lung cell populations than their PBS administered counterparts. Similar to the cellular response, the humoral response of immunized Il1a^{Δ559,1} and wildtype mice did not significantly differ upon vaccination. Both groups of immunized mice had elevated levels of OmpX specific serum IgG and lung IgA compared to their respective PBS administered controls (FIGS. 12D-E). Though lung IgA levels of immunized Il1a^{Δ559,1} mice trended to being higher than those of immunized wildtype mice, the differences were not statistically significant (FIG. 12E). When examining bacterial burdens in the lung and spleen following pulmonary challenge with K. pneumoniae, we found that both immunized Il1a^{Δ559,1} and wildtype mice had reduced burdens in the lungs and spleens compared to PBS treated mice (FIGS. 12F-G).

Given that Il1a^{Δ559,1} may not result in complete absence of IL-1α, we tested serum levels of IL-1α before and 24 hours after pulmonary challenge with K. pneumoniae in knockout and wildtype mice. Additionally, 24 hours post-challenge, we collected and homogenized the lungs of Il1a^{Δ559,1} and wildtype mice and tested the supernatant for IL-1α using ELISA. As expected, the Il1a^{Δ559,1} mice had no detectable IL-1α in the serum prior to infection, and a small, though not statistically significant, increase 24 hours after infection. Conversely, the wildtype mice had a dramatic rise in serum IL-1α following infection (FIG. 12H). However, when evaluating IL-1α in lung homogenate supernatant following infection, we found that both Il1a^{Δ559,1} and wildtype mice had elevated levels of IL-1α that were not significantly different from one another (FIG. 12I). These data suggest that while the Il1a^{Δ559,1} mice have significantly impaired systemic IL-1α production, this impairment does not extend to IL-1α at a tissue level. The inability for this mouse model to deplete IL-1α in the lungs offers an explanation as to why we were unable to replicate the phenotype of reduced numbers of Th17 cells in the lungs of immunized IL1R1−/− with the Il1a^{Δ559,1} mice.

While we were unable to find a true IL-1α deficient genetic model, we next used antibody neutralization to investigate the role of IL-1α in enhancing the tissue resident Th17 population. To neutralize IL-1α, wildtype mice immunized as described were injected interperitoneally with anti-IL-1α, anti-IL-1β, or isotype control antibodies one day prior to receiving their 2nd vaccine dose. Following challenge as above, we observed that neutralization of IL-1 signaling had no impact on the numbers of CD4+ T cells and B cells in the lungs (FIG. 10A-B). However, treatment with anti-IL-1α or anti-IL-1β resulted in a significant reduction in the number of lung Th17 cells compared to isotype control-treated mice (FIG. 10B). Of all the treatment groups, antibody mediated neutralization of IL-1α alone led to the most significant reduction in Th17 cells. These data support our observations in FIG. 8 demonstrating a role for IL-1R1 signaling in vaccine-mediated Th17 cell production. Analysis of whole lung single cell suspensions using IL-17A ELISpot revealed there was no difference in the number of IL-17A secreting cells from vaccinated mice regardless of antibody treatment (FIG. 12D). Given these data represent the whole lung, including γδ T cells and/or type 3 innate lymphoid cells (ILC3s) which also secrete IL-17A, differences in IL-17A production from Th17 cells could be obscured. In addition, mice treated with IL-1α or IL-1β neutralizing antibodies had levels of OmpX specific serum IgG comparable to vaccinated mice treated with isotype control antibody, suggesting inhibition of IL-1 signaling had no impact on vaccine elicited antibody responses (FIGS. 10E-F).

We next evaluated how neutralization of IL-1 signaling influenced protective efficacy of our vaccine. Upon examining lung burdens of challenged mice, we found that treatment with anti-IL-1α resulted in significantly higher bacterial burdens than the isotype-treated vaccinated mice and comparable burdens to unvaccinated naïve mice (FIG. 10G). Interestingly, vaccinated mice treated with IL-1β neutralizing antibody retained a level of protective efficacy with significantly lower lung burdens than the naïve mice and no significant difference from isotype-treated vaccinated mice. The evaluation of spleen bacterial burdens demonstrated a more dramatic effect of IL-1 neutralization on bacterial dissemination. Similar to the lungs, neutralization of IL-1α signaling eliminated the protective efficacy of our vaccine and there was no significant difference between naïve mice and anti-IL-1α-treated vaccinated mice (FIG. 10H). Unlike our observations with lung burdens, vaccinated mice treated with IL-1β neutralizing antibody had significantly higher spleen burdens than the vaccinated isotype treated controls.

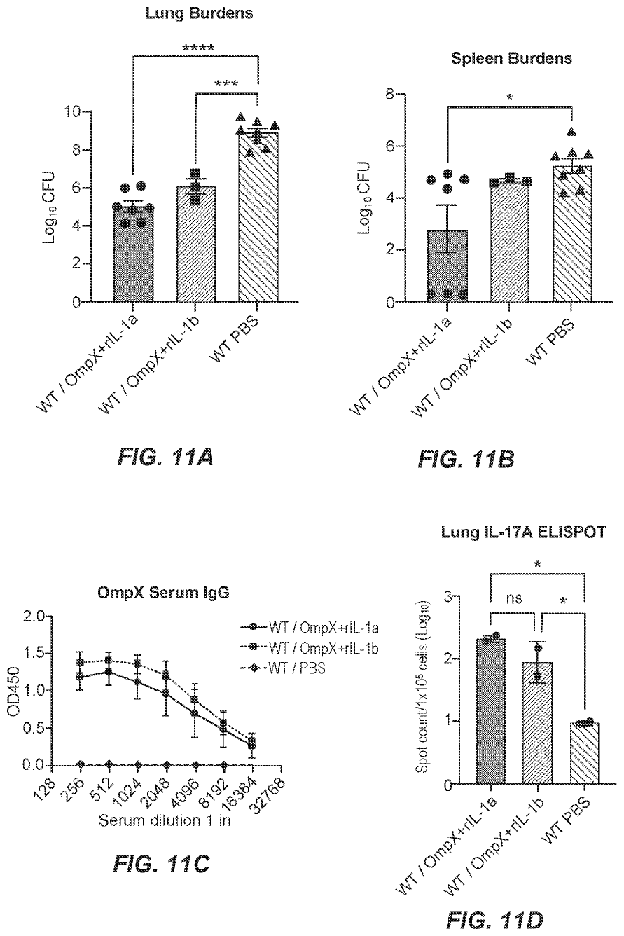

IL-1α as a vaccine adjuvant generates a protective immune response. Since our data demonstrate that IL-1-mediated signaling influences generation of Th17 cells and protection in our vaccine model, we next examined whether IL-1α or IL-1β could be used as a vaccine adjuvant to confer immunity and protection in our model. To test this, we immunized mice following the previously described schedule replacing the LTA1 adjuvant with either recombinant IL-1α or IL-1β. Upon challenging vaccinated mice with K. pneumoniae, we found that mice adjuvanted with either rIL-1α or rIL-1β had significantly reduced lung bacterial burdens compared to PBS administered controls, with the rIL-1α and rIL-1β groups reducing the burdens by 4 and 3 logs respectively (FIG. 11A). Protection from dissemination, as indicated by bacterial burdens in the spleen, were not as clear. There was no difference between IL-1β adjuvanted mice and PBS controls. Additionally, 3 out of 7 of the IL-1α adjuvanted mice were protected from bacterial dissemination as evidenced by having no detectable bacteria in the spleen, though differences between PBS administered and rIL-1α adjuvanted groups were not significant (FIG. 11B). Further, we found that rIL-1α and rIL-1β as adjuvants were sufficient in generating adaptive immune responses. Both vaccination groups generated high levels of OmpX-specific serum IgG and elevated lung IL-17A-secreting T cells when compared with the PBS administered group, as evidenced by ELISA and ELISpot, respectively (FIGS. 11C-D).

### Example 10

This Example discusses the results presented in the preceding Example.

We have demonstrated that signaling through IL-1R plays a major role in vaccine-mediated lung Th17 TRM generation. Analysis of sc-RNA-seq data revealed that these cells express high levels of IL-1R1 compared to naïve CD4+ T cells. Cytokine analysis in the BAL of immunized mice showed vaccination induced both IL-1α and IL-1β. Somewhat surprisingly, vaccine driven lung Th17 cells were generated independent of caspase-1, demonstrating that inflammasome cleavage of IL-1β or IL-18 is dispensable. However, neutralization of IL-1α was associated with reduced lung Th17 TRM generation. Moreover, recombinant IL-1α was somewhat more effective than IL-1β as a cytokine adjuvant to generate lung Th17 cells. Taken together, these data suggest that IL-1α is a major downstream event for LTA1 to induce lung Th17 TRM cells, and show that IL-1α can serve as a cytokine adjuvant to generate these types of immune responses in the lung.

Elucidation of mechanisms underlying Th17 TRM development from naïve T cells is critical for design and implementation of new mucosal adjuvants. The results reported herein demonstrate that an LTA1 adjuvanted vaccine activates IL-1 signaling pathways to elicit Th17 TRM cells. These data are consistent with previous reports suggesting parenterally administered LT also elicits Th17 cells via signaling through IL-1R1. While previous studies have shown LTA1 activates NLRP3, cleavage of pro-caspase-1 into caspase-1, and secretion of IL-1β in vitro, IL-1β appeared to be dispensable for Th17 cell generation in our model. Instead, we found that our LTA1 adjuvanted vaccine acted through IL-1α signaling, in part, to promote antigen-specific Th17 cells, in line with previously demonstrated functions of IL-1α in promoting activation and expansion of memory T cells. Indeed, antibody neutralization of IL-1α resulted in a decrease of vaccine-induced Th17 cell generation coupled with a reduction in vaccine-mediated protection. However, animal models knocking down IL-1α expression in vivo failed to demonstrate a phenotype in our studies. We expect this is due to incomplete suppression of IL-1α release in Casp1/4^{-/-} and Il1a^{Δ559,1} mice, where we observed similar levels of K. pneumoniae-induced IL-1α in the lungs of wildtype and Il1a^{Δ559,1} mice. These results suggest even low levels of IL-1α could promote Th17 cell formation. Given we did not observe a complete abrogation of Th17 production upon neutralization of IL-1α it is possible this cytokine is not the only factor facilitating development of Th17 TRMs from naïve T cells. Thus, the roles additional cytokines or other signaling pathways play in vaccine-mediated elicitation of memory T cells remain to be determined.

That IL-1β appears to be dispensable for Th17 cell development following immunization was somewhat unexpected given previous studies demonstrating a primary role for IL-1β in Th17 cell development in mice and humans. In addition, the LTA1-related adjuvants LT and double-mutant LT (dmLT) have been shown to stimulate Th17 cells through IL-1β and IL-23 related mechanisms. Somewhat contradictory to our animal data, antibody neutralization of IL-1β resulted in a reduction in lung Th17 cell generation following vaccination, though these mice still had reduced burdens in the lung following K. pneumoniae challenge. One possible explanation is that IL-1α, but not IL-1β, is necessary to promote protective effector cytokine production (i.e., IL-17A, IL-17F, and IL-22) from vaccine-elicited Th17 cells (11).

Our results show IL-1α is an effective mucosal adjuvant for eliciting both cellular and humoral responses to GNB vaccine antigens in vivo (FIGS. 11A-D). Importantly, the humoral response elicited by the exemplar vaccine formulation tested was a Th17 cell response. As persons of skill will appreciate, the humoral portion of the body's immune response to allergens, self-cells, and pathogens involves the polarization of T-helper type 1 (Th1), Th2, or Th17 cells. The polarization of T cells into one of these three phenotypes is “determined by the complex interaction of antigen-presenting cells with naïve T cells and involves a multitude of factors, including the dominant cytokine environment, costimulatory molecules, type and load of antigen presented and a plethora of signaling cascades.” See, Abstract, Kaiko, et al., Immunology, 2008, 123(3):326-338; doi: 10.1111/j.1365-2567.2007.02719.x. Further, as noted by Kaiko et al.: “many different elements act synergistically, antagonistically and through positive feedback loops to activate a Th1, Th2, or Th17 immune response.” Id. Thus, factors that polarize T helper cells along the Th1 or Th2 pathway do not polarize them along the Th17 pathway important for a humoral response to bacterial pathogens, such as Gram-negative organisms in general, or Enterobacteriaceae in particular.

Identification of adjuvants that elicit Th17 cells is critical to development of more broadly effective vaccines against GNB. The discovery that IL-1α is important for eliciting Th17 cells is thus an important advance. It is also a surprising advance. Prior reports studying the eliciting of IL-1 cytokines or their use as adjuvants showed results that indicated the agents polarized T cells to become Th1 or Th2 cells, rather than Th17 cells, and thus indicated that IL-1α would not be useful as an adjuvant for vaccines against these bacterial pathogens.

For example, Matsushita and Yoshimoto (J Immunol., 2014, 193(12):5791-800) administered ragweed pollen into the lungs of mice and found that ragweed pollen both elicited production of “IL-1α/β” and activated production of IgE. It is known that IgE production requires IL-4 produced by Th2 cells (see, e.g., Yanagihara, et al., J Allergy Clin Immunol, 1995, 96(6 Pt 2):1145-51), and the Matsushita and Yoshimoto thus indicated that the administration of an allergen produces IL-4, suggesting that T cells were polarized along the Th2 pathway in the presence of (or despite the presence of) IL-1α/β. Further, IL-4 suppresses Th17 cells. See, e.g., Harrington, et al., Nat Immunol, 2005, 6(11):1123-32; doi: 10.1038/ni1254. Thus, the Matsushita and Yoshimoto report would suggest that the presence of IL-1 cytokines not only did not polarize T cells to become Th17 cells, but that the production of Th17 cells was repressed in their presence.

A study of IL-1β as a mucosal adjuvant in an influenza vaccine reported that the authors did not find elevated levels of IL-17-producing CD4+ T cells, “indicating no or only minor impact of IL-1β expression on the induction of T_{H}17 cells.” (See, Lapuente, et al. Mucosal Immunol., 2018, 11(4):1265-78). Thus, the tissue-resident memory cells reported by the authors in that study were not Th17 cells.

An influenza vaccine adjuvanted with IL-1α was reported to result in high levels of Th1-type cytokines, which adjuvanting with IL-1β was reported to result in high levels of Th2-type cytokines, (see, Kayamuro, et al. J Virol. 2010, 84(24):12703-12), suggesting that neither IL-1 cytokine provided the Th17 cell response needed for protection against a bacterial GNB pathogen. A report on the adjuvant effect of IL-1α administered subcutaneously was shown to mildly enhance T helper cell generation in vivo (see, Khoruts, et al., Eur J Immunol. 2004, 34(4):1085-90), but the authors reported they “could not detect significant production of any measured pro-inflammatory cytokines in response to IL-1 by any DC subsets.” (IL-17, which is produced by Th17 cells, is a pro-inflammatory cytokine.)

The work reported here demonstrates, in contrast to the reports summarized above, that IL-1α as a mucosal adjuvant can elicit both antigen-specific antibodies and lung Th17 TRMs when admixed with an exemplar GNB Omp, OmpX, providing protection against subsequent K. pneumoniae challenge.

One limitation of our study is that while IL-1R1 appears to be important for eliciting vaccine-induced protective Th17 cells, it does not appear to be the only factor leading to Th17 cell production. Elucidating additional pathways leading to vaccine-mediated Th17 cells will further improve mucosal vaccine development. Importantly, Th17 cells produced in vaccinated Il1r1^{-/-} mice still provided protection upon subsequent challenge, so a future focus will be to determine whether T cells generated in wild-type and Il1r1^{-/-} mice are functionally different. In addition, we do not know the longevity of Th17 cells elicited by an IL-1α adjuvant. Thus, future studies will examine the lifetime of these cells in immunized mice to determine the duration of the protection they provide.

Overall, we have demonstrated IL-1α signaling is important for vaccine-mediated generation of Th17 TRMs in mice. In addition, IL-1α and, to a lesser extent, IL-1β appear to be effective on their own to generate these cells when admixed with the exemplar K. pneumoniae antigen OmpX. Our study sheds light on mechanisms by which LTA1 induces vaccine-mediated protection and further highlights the potential of IL-1 cytokines as mucosal vaccine antigens. Overall, the increased understanding of the adjuvant-dependent mechanisms through which naïve T cells develop into Th17 TRM cells provided by the studies herein not only provide an improved mucosal vaccine against GNB, within GNB, Enterobacteriaceae and, within Enterobacteriaceae, K. pneumoniae in particular, but will also inform development of mucosal vaccines against other pathogens.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. An immunogenic composition, said composition comprising (a) at least one immunogen from a Gram-negative bacterium (“GNB”) and (b) IL1α.

2. The immunogenic composition of claim 1, wherein said GNB is an Enterobacteriae.

3. The immunogenic composition of claim 1, wherein said GNB is Klebsiella pneumoniae.

4. (canceled)

5. The immunogenic composition of claim 1, further comprising an Omp from a second GNB.

6. The immunogenic composition of claim 1, wherein said at least one immunogen from said GNB and said IL1α are in a fusion protein.

7. (canceled)

8. The immunogenic composition of claim 1, wherein said at least one immunogen from said GNB is an outer membrane protein (“Omp”) or two or more different Omps.

9. (canceled)

10. The immunogenic composition of claim 8, wherein said Omp is from K. pneumoniae.

11. The immunogenic composition of claim 10, further wherein said K. pneumoniae Omp is one or more of OmpX, OmpC, OmpW, and Omplolb.

12. (canceled)

13. The immunogenic composition of claim 11, further wherein said immunogenic composition is a fusion protein comprising (a) one or more of OmpX, OmpC, OmpW, and Omplolb, and (b) IL1α.

14-16. (canceled)

17. The immunogenic composition of claim 13, further wherein said fusion protein comprises OmpX and IL1α.

18. (canceled)

19. The immunogenic composition of claim 18, wherein said fusion protein has the sequence of SEQ ID NO:1.

20. A method of increasing a subject's immune response to an immunogen from a Gram-negative bacterium (“GNB”), said method comprising co-administering to said subject (a) an effective amount of said immunogen and (b) an effective amount of IL1α.

21-24. (canceled)

25. The method of claim 20, wherein said GNB immunogen is from an Enterobacteriae bacterium.

26. The method of claim 20, wherein said GNB immunogen is from Klebsiella pneumoniae.

27. (canceled)

28. The method of claim 20, wherein said GNB immunogen is an outer membrane protein (“Omp”) or two or more different Omps.

29. (canceled)

30. The method of claim 28, further wherein said Omp is from K. pneumoniae, or said two or more Omps are from K. pneumoniae, and said K. pneumoniae Omp or Omps are one or more of OmpX, OmpC, OmpW, and Omplolb.

31. The method of claim 30, further wherein said coadministration of one or more of OmpX, OmpC, OmpW, and Omplolb and IL1α is by administering a fusion protein of (a) one or more of OmpX, OmpC, OmpW, and Omplolb and (b) IL1α.

32-33. (canceled)

34. The method of claim 30, wherein said Omp is OmpX.

35. The method of claim 31, further wherein said coadministration is of OmpX and IL1α.

36. (canceled)

37. (canceled)

38. The method of claim 35, wherein said fusion protein has the sequence of SEQ ID NO:1.

39-41. (canceled)


SEQUENCE LISTING (9 sequences)

SEQ ID NO: 1 - protein, 316 aa, synthetic construct
MGSGHHHHHHGGSMTGGYAQSDMQGKANKAGGFNLKYRYEQDNNPLGVIGSFTYTEKDNTSNGTYNKGQYYGITAGPAYRLNDWASIYGVVGVGYGKFQNNNFPNQKSDMSDYGFSYGAGLQFNPIENVALDFSYEQSRIRNVDVGTWIAGVGYRFGGGGSAPYTYQSDLRYKLMKLVRQKFVMNDSLNQTIYQDVDKHYLSTTWLNDLQQEVKFDMYAYSSGGDDSKYPVTLKISDSQLFVSAQGEDQPVLLKELPETPKLITGSETDLIFFWKSINSKNYFTSAAYPELFIATKEQSRVHLARGLPSMTDFQIS

SEQ ID NO: 2 - protein, 5 aa, synthetic construct
GGGGS

SEQ ID NO: 3 - protein, 4 aa, synthetic construct
IEGR

SEQ ID NO: 4 - protein, 6 aa, synthetic construct
LVPRGS

SEQ ID NO: 5 - protein, 4 aa, synthetic construct
AAAA

SEQ ID NO: 6 - protein, 5 aa, synthetic construct
AAAAA

SEQ ID NO: 7 - protein, 10 aa, synthetic construct
GGGGSGGGGS

SEQ ID NO: 8 - protein, 15 aa, synthetic construct
GGGGSGGGGSGGGGS

SEQ ID NO: 9 - protein, 20 aa, synthetic construct
GGGGSGGGGSGGGGSGGGGS